(19) United States
(12) Patent Application Publication
(10) Pub. No.: US 2008/0146495 A1
(43) Pub. Date: Jun. 19, 2008

(54) Methods for Treating Hiv by Inhibiting Cd4 Down-Modulation

(75) Inventor: Juan Lama, San Diego, CA (US)

Correspondence Address: BIOTACTICA, LLC, 9909 Manchester Road, PMB 322, ST. LOUIS, MO, 63122-1915, US

(73) Assignee: The Regents of the University of California, Oakland, CA (US)

(21) Appl. No.: 11/908,378

(22) PCT Filed: Mar. 11, 2005
(86) PCT No.: PCT/US2005/008241

§ 371 Date: Sep. 19, 2007

(51) Int. Cl. A61K 38/00; A61K 31/70; G01N 33/53; C12N 15/00

(52) U.S. Cl. 514/8; 514/44; 435/7.2; 435/7.9; 435/320.1

(57) ABSTRACT

The present invention provides methods for inhibiting the ability of HIV genes products to bind to the cytoplasmic domain of a CD4 receptor. This domain is necessary to the ability of the HIV Nef and Vpu gene products to connect the CD4 receptor to cellular degradation pathways. The present invention provides a method for introducing a modified CD4 receptor into a cell, and inhibitors thereof, to treat HIV infection and CD4 receptor down-modulation. The present invention is also directed to a method for screening compounds for the ability to inhibit CD4 down-modulation.

### STATEMENT REGARDING FEDERALLY SPONSORED RESEARCH OR DEVELOPMENT

This invention was made in part with Government support under National Institutes of Health Grants R01-DA13866 and R01-AI058851. The Government has certain rights in the invention.

### CROSS-REFERENCE TO RELATED APPLICATIONS

Not Applicable.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND OF THE INVENTION

1. Field of the Invention

The present invention relates generally to a method for treating, preventing, reversing or inhibiting HIV infection, and more specifically to a method for treating (which includes preventing, reversing or inhibiting) HIV infection by inhibiting the ability of HIV to down-modulate CD4. The present invention also relates to a method for screening inhibitors of HIV-induced CD4 down-modulation.

2. Description of Related Art

Viral infection of cells is generally initiated through the binding of viral envelope proteins to specific receptors on the cell surface. After binding to the cell receptor, the virus enters the cell either through fusion of the viral envelope with the cell membrane or by receptor-mediated endocytosis. The CD4 cell surface receptor is a high-affinity receptor for Human immunodeficiency viruses (HIV-1 and HIV-2) and Simian immunodeficiency virus (SIV). Indeed, CD4 is the primary cellular receptor for each of these viruses. HIV infection of CD4-positive cells is initiated by the binding of HIV outer envelope glycoprotein gp120 to CD4.

CD4 plays a dual role during HIV infection. The cell surface receptor is required for entry of HIV into most permissive cells. During late stages of infection, however, the receptor exerts inhibitory effects on the infectivity of the released particles. To overcome these effects, HIV has evolved mechanisms that ensure the removal of the CD4 receptor from the surface of infected cells. These mechanisms are known generally as CD4 down-modulation.

Three viral proteins participate in the process of CD4 down-modulation in HIV-1: Nef, Vpu, and Env. The effects of Nef (early product) and Vpu/Env (late products) are quantitative and qualitatively distinct. Nef enhances CD4 internalization from the cell surface and targets the receptor for degradation into lysosomes, whereas Env interferes with transport of CD4 to the cell surface and Vpu targets CD4 for degradation in proteasomes. However, to date, no specific inhibitors of the HIV-induced CD4 down-modulation, including Nef, Vpu or Env binding with CD4, have been characterized.

Therefore, what is needed, is a therapeutic method for treating HIV infection by providing to subjects in need thereof inhibitors of HIV-induced down-modulation of the CD4 receptor. What is also needed is a method of identifying compounds that interfere with HIV-induced down-modulation of CD4.

### BRIEF SUMMARY OF THE INVENTION

Accordingly, it is an object of the invention to overcome these and other problems in the art. These and other objects, features, and technical advantages are achieved by inhibiting or attenuating the ability of HIV to down-modulate CD4 receptor.

This invention provides a method for treating HIV infection comprising administering a therapeutically effective amount of a CD4 receptor down-modulation inhibitor to a subject in need thereof. In one aspect of the invention, the inhibitor is CD4Δcyt. The CD4Δcyt can comprise a polypeptide sequence corresponding to SEQ ID NO:1. The CD4Δcyt can also be a gene product of a vector operably linked to a polynucleotide encoding CD4Δcyt and wherein the subject is a mammal suffering from HIV infection or susceptible to HIV infection. The polynucleotide encoding CD4Δcyt can comprise a sequence corresponding to SEQ ID NO:2.

In accordance with a further aspect of the invention, the inhibitor can be a CD4 cytoplasmic domain, fragment, or variant thereof. The CD4 cytoplasmic domain can be a gene product of a vector operably linked to a polynucleotide encoding a CD4 cytoplasmic domain and wherein the subject is a mammal suffering from HIV infection or susceptible to HIV infection. The inhibitor can also be a gene product of a vector operably linked to polynucleotides encoding a CD4 protein lacking a CD4 cytoplasmic domain. The inhibitor can also be a gene product of a vector operably linked to polynucleotides encoding a CD4 extracellular domain, a CD4 transmembrane domain, and a HIV Matrix protein.

In various aspects on the invention, the vector can be selected from the group consisting of an adenoviral vector and a lentiviral vector. The vector can also be selected from the group consisting of PPT-PGK-CD4Δcyt, pHR′-CMV-CD4Δcyt-WPRE, and pWPI-CD4Δcyt. In various aspects, the subject can be a human.

In various aspects of the invention, the inhibitor can be a CD4 receptor cytoplasmic domain-specific polypeptide, oligonucleotide, probe, antibody, protein-methylation compound, ribozyme, siRNA, triplex-forming oligonucleotide, and antisense molecule. In one aspect, the inhibitor prevents at least a portion of a cytoplasmic domain of said CD4 receptor from contacting an HIV gene product. In another aspect, the inhibitor prevents a chemokine receptor from contacting an HIV gene product. The HIV gene product can be selected from the group consisting of Nef, Env, Vpu, and combinations thereof. The chemokine receptor can be selected from the group consisting of CCR5 and CXCR4.

In accordance with another aspect of the invention, a method is provided for inhibiting CD4 receptor down-modulation, comprising contacting a HIV-infected mammalian cell with CD4Δcyt. In another aspect of the invention, a method is provided for inhibiting CD4 receptor down-modulation, comprising contacting a CD4 receptor cytoplasmic domain with a composition selected from the group consisting of a CD4 receptor cytoplasmic domain-specific polypeptide, oligonucleotide, probe, antibody, protein-methylation compound, ribozyme, siRNA, triplex-forming oligonucleotide, and antisense molecule.

In accordance with yet another aspect of the invention, a method is provided for identifying the activity of an inhibitor of HIV-induced down-modulation of a CD4 receptor comprising: providing a plurality of CD4-positive cells, said CD4-positive cells being infected with HIV; providing a target compound to be assayed for activity in inhibiting HIV-induced CD4 down-modulation; incubating said CD4-positive cells in the presence of said target compound; providing a plurality of HIV-1 Env-positive cells; incubating said HIV-1 Env-positive cells with said CD4-positive cells and said target compound; and measuring the level of fusion between said HIV-1 Env-positive cells and said CD4-positive cells, whereby a relatively higher measured level of fusion is indicative of a relatively higher activity of the target compound as an inhibitor of HIV-induced down-modulation.

In one aspect, the CD4-positive cells express a beta-lactamase enzyme, and further wherein said HIV Env-1 positive cells are labeled with a substrate of beta-lactamase. In another aspect, the extent of fusion between said CD4-positive cells and said HIV-1 Env-positive cells is determined by analyzing the extent of cleavage of said substrate of beta-lactamase. In yet another aspect, the substrate of beta-lactamase is CCF2-AM.

In accordance with yet another aspect of the invention, a polynucleotide is provided encoding a CD4 protein having a nonfunctional CD4 cytoplasmic domain. The nonfunctional cytoplasmic domain can be a truncated CD4 cytoplasmic domain. In another aspect, the CD4 protein comprises a nucleotide sequence corresponding to SEQ ID NO:2. In yet another aspect, a polynucleotide is provided encoding a CD4 protein lacking a CD4 cytoplasmic domain.

In accordance with a further aspect of the invention, a vector is provided operably linked to a polynucleotide encoding a CD4 protein having a nonfunctional cytoplasmic domain. The nonfunctional CD4 cytoplasmic domain can be a truncated CD4 cytoplasmic domain. In another aspect, a vector is provided operably linked to a polynucleotide encoding a CD4 protein lacking a cytoplasmic domain. In various aspects of the present invention, the vector can be selected from the group consisting of an adenoviral vector and a lentiviral vector. The lentiviral vector can be selected from the group consisting of PPT-PGK-CD4Δcyt, pHR′-CMV-CD4Δcyt-WPRE, and pWPI-CD4Δcyt. In various aspects, the encoded CD4 protein comprises a nucleotide sequence corresponding to SEQ ID NO:2.

In accordance with another aspect of the invention, an isolated polypeptide is provided comprising a nonfunctional CD4 cytoplasmic domain. In various aspects, the nonfunctional CD4 cytoplasmic domain comprises a deletion of at least one amino acid. In one alternative, the nonfunctional CD4 cytoplasmic domain comprises an insertion of at least one amino acid. In another aspect, the polypeptide comprises an amino acid sequence corresponding to SEQ ID NO:1. In another aspect of the invention, an isolated polypeptide is provided comprising a CD4 extracellular domain, a CD4 transmembrane domain, and an HIV Matrix protein. In another aspect, an isolated polypeptide is provided comprising a CD4 protein lacking a cytoplasmic domain.

In accordance with yet another aspect of the invention, a kit is provided for screening compounds for inhibition of HIV-induced down-modulation of CD4 comprising: a plurality of CD4-positive cells, said CD4-positive cells infected with HIV; a plurality of HIV-1 Env-positive cells; and a buffer in which a compound to be screened for inhibition of HIV-1 induced CD4 down-modulation can be incubated in the presence of said CD4-positive cells, and further in which said HIV-1 Env-positive cells can be incubated in the presence of said compound to be screened and said CD4-positive cells. In various aspects, the kit may further comprising a detection system for detecting the extent of fusion between said CD4-positive cells and said HIV-1 Env-positive cells, said detection system being selected from the group consisting of electrical measurement systems, fluorescent labeling systems, automated cell sorting systems, microscopy systems, fluorescent microscopy systems, spectrophotometric systems, chromatographic systems, enzyme detection systems, energy transfer systems, and combinations thereof. In another aspect, the CD4-positive cells express a beta-lacatamase enzyme, and further wherein said HIV-1 Env-positive cells comprise a substrate of beta-lactamase. In yet another aspect, the kit further comprises a detection system for detecting the extent of cleavage of said substrate of beta-lactamase. In various aspects, the kit may further comprise a detection system for determining the extent of cleavage of CCF2-AM.

In yet another aspect of the present invention, a mammalian cell is provided comprising one of the polynucleotides, the vectors, or the polypeptides as provided above. In another aspect, a mammal is provided comprising a cell as provided above.

These and other features, aspects and advantages of the present invention will become better understood with reference to the following description, examples and appended claims.

### BRIEF DESCRIPTION OF THE SEVERAL VIEWS OF THE DRAWINGS

FIG. 1. is a schematic drawing of the lentiviral vectors PPT-PGK-GFP and pHR′-CMV-GFP. These vectors were used to express GFP alone (GFP), full-length CD4 (CD4WT), CD4 lacking its cytoplasmic domain (CD4Δcyt), or the extracellular and transmembrane domain of CD4 fused to HIV-1 Matrix (CD4-MA).

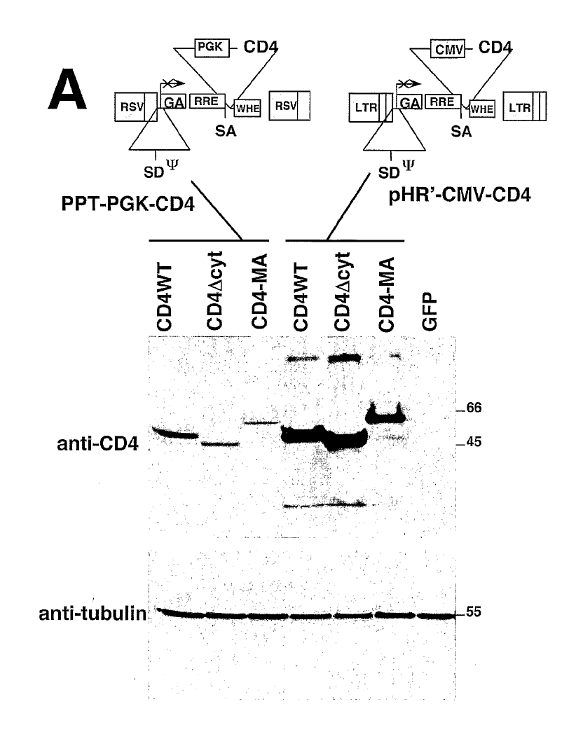

FIG. 2. 293T cells were transduced with pHR′CMV vectors expressing either wild-type, CD4-MA, or CD4Δcyt and transfected with a plasmid expressing Nef (NA7) fused to GFP. Histograms represent CD4 levels in GFP-positive cells. IgG isotypic control is shown as dotted lines.

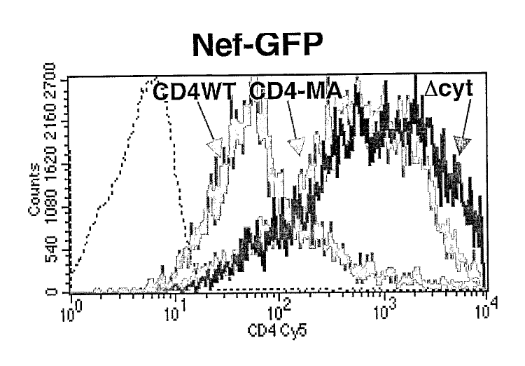

FIG. 3. 293T cells were transduced with pHR′CMV vectors expressing either wild-type, CD4-MA, or CD4Δcyt and transfected with a plasmid expressing GFP. Histograms represent CD4 levels in GFP-positive cells. IgG isotypic control is shown as dotted lines.

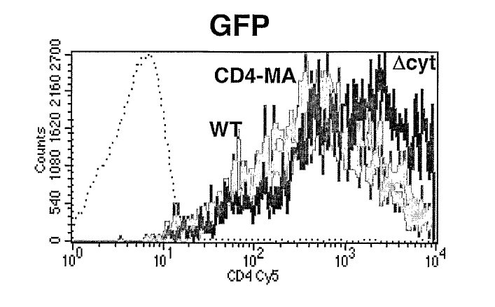

FIG. 4. Estimates of the levels of HIV-1 p24 antigen in 293T cell culture supernatants 48 hours after transfection.

FIG. 5. 24 hours after transduction with pHR′-CMV lentiviral vectors expressing CD4 variants, 293T cells were either mock-infected (left columns), infected with HIV-1 R9 (middle columns), or with HIV-1 R9 pseudotyped with VSV G (right columns).

FIG. 6. Infectivity of the released particles in MAGIC5 cells infected with equal amounts of p24 input virus. Control infection was performed with virus produced in 293T cells infected with HIV-1 pseudotyped with VSV G. The effect of addition of 1 M AZT before infection of MAGIC5 cells with HIV-1 (VSV) is also shown.

FIG. 7. CD4-positive MAGIC5B, and C8166 cells were mock-treated, or transduced, with lentiviral vectors (pHR′-CMV) expressing either full-length CD4 or truncated CD4. CD4-positive SupT1 cells were transduced with PPT-PGK vectors. 24 hours after transduction cells were infected with HIV-1 (R9). The extent of CD4 down-modulation in HIV-infected cells (p24-positive) was analyzed 48 h after infection, as shown in FIGS. 5-6. Left and right numbers indicate the mean CD4 values in uninfected and infected (gray dots) cells, respectively.

FIG. 8. infectivity of the released particles estimated in MAGIC5 cells, as shown in FIG. 5.

FIG. 9. PBMC were mock-transduced with lentiviral vectors (pWPI). The PBMC were then mock-treated and stained for surface CD4. Inlet numbers represent the percentage of transduced cells with high or low levels of surface CD4 (upper and lower numbers, respectively).

FIG. 10. PBMC were transduced with lentiviral vectors (PWPI) expressing GFP and full-length CD4. Transduced PBMC were then mock-treated and stained for surface CD4. Inlet numbers represent the percentage of transduced cells with high or low levels of surface CD4 (upper and lower numbers, respectively).

FIG. 11. PBMC were transduced with lentiviral vectors (PWPI) expressing GFP and full-length CD4. Transduced PBMC were then infected for 48 hours with HIV-1 and stained for surface CD4. Inlet numbers represent the percentage of transduced cells with high or low levels of surface CD4 (upper and lower numbers, respectively).

FIG. 12. PBMC were transduced with lentiviral vectors (PWPI) expressing GFP alone. Transduced PBMC were then mock-treated and stained for surface CD4. Inlet numbers represent the percentage of transduced cells with high or low levels of surface CD4 (upper and lower numbers, respectively).

FIG. 13. PBMC were transduced with lentiviral vectors (PWPI) expressing GFP and CD4Δcyt. Transduced PBMC were then mock-treated and stained for surface CD4. Inlet numbers represent the percentage of transduced cells with high or low levels of surface CD4 (upper and lower numbers, respectively).

FIG. 14. PBMC were transduced with lentiviral vectors (PWPI) expressing GFP and CD4Δcyt. Transduced PBMC were then infected for 48 hours with HIV-1 and stained for surface CD4. Inlet numbers represent the percentage of transduced cells with high or low levels of surface CD4 (upper and lower numbers, respectively).

FIG. 15. PBMC were transduced with lentiviral vectors (PWPI) expressing GFP and full-length CD4. Transduced PBMC were then infected for 48 hours with HIV-1 and stained for intracellular p24. Inlet numbers represent the percentage of GFP-positive/p24-positive cells.

FIG. 16. PBMC were transduced with lentiviral vectors (pWPI) expressing GFP and CD4Δcyt. Transduced PBMC were then infected for 48 hours with HIV-1 and stained for intracellular p24. Inlet numbers represent the percentage of GFP-positive/p24-positive cells.

FIG. 17. PBMC cells were transduced with pWPI lentiviral vectors expressing either GPF alone, together with CD4, or together with CD4Δcyt. Cultures were enriched with transduced cells by sorting GFP-positive/CD4-positive cells in a flow cytometry apparatus. Mock-transduced cells are shown for comparison. Numbers indicate the percentage of CD4-positive/GFP-positive cells.

FIG. 18. Three days after sorting, cell described in FIG. 17 were infected with CCR5-tropic HIV-1 BaL strain. Viral replication was estimated by measuring accumulation of p24 protein in cultures.

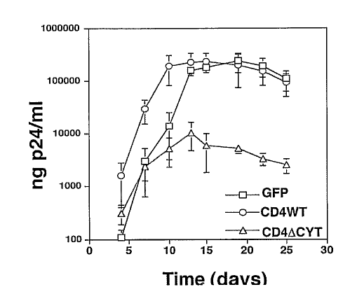

FIG. 19. Jurkat T cells were transduced with pWPI vectors and analyzed by flow cytometry with CD4-specific antibody (without further enrichment).

FIG. 20. Jurkat T cells were infected with the CXCR4-tropic R9HIV-1 strain and the extent of viral replication estimated by measuring accumulation of p24 protein in cultures.

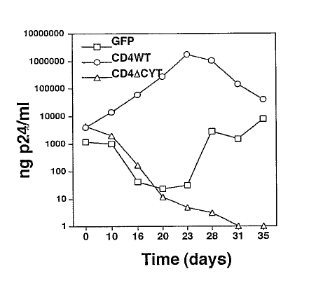

FIG. 21. 293T cells previously transduced with lentiviral vectors (pHR′-CMV) expressing either GFP or CD4 variants were infected with HIV-1 pseudotyped with VSV G. 48 hours post-infection, culture supernatants were collected and used to purify virions through sucrose cushions. Cell lysates from producer cells (lower panels) and purified virions (upper panels) were analyzed by SDS/PAGE/western blot with antibodies specific to CD4, gp120, gp41 and p24. Molecular weights in kDa are shown. Left-bottom numbers indicate the amount of HIV-1 particles released from infected cells (estimated by p24 ELISA), and their infectivity estimated in MAGIC5 cells. Values are given as percentage of those observed in virions from cells transduced with the pHR′-CMV-GFP control vector.

### DETAILED DESCRIPTION OF THE INVENTION

### Abbreviations and Definitions

Unless otherwise defined, all technical terms used herein have the same meaning as commonly understood by one of ordinary skill in the art to which this invention belongs. Commonly understood definitions of molecular biology terms can be found in Rieger et al., GLOSSARY OF GENETICS: CLASSICAL AND MOLECULAR, 5^{th} edition, Springer-Verlag; New York, 1991; and Lewin, Genes V, Oxford University Press: New York, 1994. To facilitate the understanding of the invention, a number of terms and abbreviations as used herein are defined below as follows:

Bind(s) or Interacts With: As used herein, the terms “bind,” or “interacts with” refers to an activity wherein one molecule recognizes and adheres to a particular second molecule in a sample, but does not substantially recognize or adhere to other structurally unrelated molecules in the sample. Generally, a first molecule that “specifically binds” to a second molecule has a binding affinity greater than about 10⁵ to 10⁶ moles/liter for that second molecule.

CD4 Receptor Down-Modulation Inhibitor: As used herein, the term “CD4 receptor down-modulation inhibitor” refers to any compound or molecule that, when administered to a subject in need thereof, results in a net increase in cellular CD4 levels, a net decrease in the number of CD4 receptors being down-modulated, or CD4 levels greater than that which would have been present in the absence of the compound or molecule. Modified CD4 receptors, such as CD4Δcyt, are included in this definition.

Gene: As used herein, the term “gene” refers to a nucleic acid molecule that codes for a particular protein, or in certain cases, a functional or structural RNA molecule. For example, the nef genes encode the Nef protein.

Nucleic Acid or Nucleic Acid Molecule: As used herein, the terms “nucleic acid” or “nucleic acid molecule” refer to a chain of two or more nucleotides such as RNA (ribonucleic acid) and DNA (deoxyribonucleic acid). A “purified” nucleic acid molecule is one that is substantially separated from other nucleic acid sequences in a cell or organism in which the nucleic acid naturally occurs (e.g., at least 30%, 40%, 50%, 60%, 70%, 80%, 90%, 95%, 96%, 97%, 98%, 99%, or 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules, e.g., the modified, variant and chimeric nucleic acids molecules provided below. Additionally, ribozymes, siRNAs, and antisense nucleic acid molecules are included within the scope of this definition.

Operably Linked: As used herein, the term “operably linked” refers to a first nucleic-acid sequence physically linked with a second nucleic acid sequence creating a functional relationship with the second nucleic acid sequence. For example, a promoter is operably linked to a coding sequence if the promoter affects the transcription or expression of the coding sequence. Generally, operably linked nucleic acid sequences are contiguous and, where necessary to join two protein coding regions, in reading frame.

Pharmaceutically Acceptable: As used herein, the term “pharmaceutically acceptable” means approved by a regulatory agency or authority either national (e.g., the FDA), supra-national (e.g., the EMEA) or other regulatory agency, governmental entity, ethics board or committee involved in the granting of Regulatory Approval or a state government or listed in the U.S. Pharmacopeia or other generally recognized pharmacopeia for use in animals, and more particularly in humans.

Pharmaceutically Acceptable Carrier: As used herein, the term “pharmaceutically acceptable carrier” refers to a diluent, adjuvant, excipient, or vehicle with which a composition is administered. Such carriers can be sterile liquids, such as water and oils, including those of petroleum, animal, vegetable or synthetic origin, such as peanut oil, soybean oil, mineral oil, sesame oil, tocopherols and the like, polyethylene glycols, glycerine, propylene glycol or other synthetic solvents. Water is a preferred carrier when a composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or any compound found in the Handbook of Pharmaceutical Excipients (4^{th} edition, Pharmaceutical Press) and the like. A composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier.

Pharmaceutically Acceptable Salt: As used herein, the term “pharmaceutically acceptable salt” includes those salts of a pharmaceutically acceptable composition formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the composition is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the composition is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc.

Pharmaceutically acceptable esters, when applicable, may be prepared by known methods using pharmaceutically acceptable acids that are conventional in the field of pharmaceuticals and that retain the pharmacological properties of the free form. Pharmaceutically acceptable esters include but are not limited to carboxylic acid esters RCOO-D (where D is a cationic form of a compound of this invention and where R is H, alkyl or aryl groups).

Protein or Polypeptide: As used herein, the terms “protein” or “polypeptide” mean any peptide-linked chain of amino acids, regardless of length or post-translational modification, e.g., glycosylation or phosphorylation. A “purified” or “isolated” polypeptide is one that is substantially separated from other polypeptides in a cell or organism in which the polypeptide naturally occurs (e.g., at least 30%, 40%, 50%, 60%, 70%, 80%, 90%, 95%, 96%, 97%, 98%, 99%, or 100% free of contaminants).

Recombinant: As used herein, the term “recombinant” refers to a nucleic acid molecule made by an artificial combination of two otherwise separated segments of sequence, e.g., by chemical synthesis or by the manipulation of isolated segments of nucleic acids by genetic engineering techniques.

Therapeutically Effective Amount: As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the Pharmaceutical Preparations and Methods of Administration section below.

Transformed, Transfected or Transgenic: As used herein, the terms “transformed,” “transfected,” or “transgenic” refer to a cell, tissue, or organism into which has been introduced a foreign nucleic acid such as a recombinant vector. The terms “transformed” or “transgenic” includes progeny of a cell or organism, including progeny produced from a breeding program employing such a “transgenic” cell or organism, as a parent in a cross.

Vector: As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors.”

Methods for Treating HIV by Inhibiting CD4 Down-Modulation

The present invention relates to inhibition of CD4 receptor down-modulation by HIV, and to methods for treating, preventing, inhibiting, or reversing HIV infection and/or AIDS by inhibiting HIV down-modulation of the CD4 receptor. In other aspects of the invention, methods are provided for identifying compositions capable of inhibiting CD4 receptor down-modulation in vitro and in vivo. In certain aspects of the invention, modified CD4 receptor molecules are provided which inhibit or prevent HIV down-modulation. In various embodiments, the modified CD4 receptor lacks all or part of the CD4 cytoplasmic domain. Such modified CD4 molecules can be administered to a mammal by methods of the present invention utilizing a vector having a nucleotide sequence encoding the modified CD4 receptor.

The cytoplasmic domain of the CD4 receptor is necessary to the ability of the HIV Nef and Vpu gene products to connect the CD4 receptor to cellular degradation pathways. Thus, one aspect of the present invention provides a method of introducing a modified CD4 receptor into a cell, the modified CD4 receptor having any modification to the nucleotide sequence encoding CD4 that results in an inhibition of the ability of Nef or Vpu to down-modulate CD4. In certain aspects of the invention, such modification can be at least a partial deletion or addition in the cytoplasmic domain such that Nef and Vpu cannot properly interact with the CD4 molecule.

Several cellular proteins are required for HIV-induced down-modulation of CD4. These proteins may not bind directly to the cytoplasmic domain of CD4, but instead serve to connect the cellular degradation machinery with CD4. These proteins bind either Nef or Vpu in order to carry out their functions. Thus, another aspect of the present invention is directed toward inhibiting the interaction of these cellular proteins with Nef, Vpu, or other HIV gene products, thereby inhibiting HIV-induced CD4 down-modulation.

In various aspects of the invention, a nucleic acid molecule encoding a modified CD4 receptor can be delivered to a mammal via a vector. Any suitable vector may be used, and it is contemplated that an adenovirus or a lentivirus vector is utilized. In various aspects, a lentiviral vector chosen from vectors derived from PPT-PGK-GFP, PHR′-CMV-GFP-WPRE, and pWPI is utilized. These vectors are delivered to target cells by methods known in the art and the target cells subsequently express the modified CD4 gene carried by said vector.

In addition, the present invention also provides for antibodies that interfere with the ability of HIV to down-modulate CD4 by binding to various compounds, molecules, or gene products important in the CD4 down-modulation process. For example, antibodies to the CD4 cytoplasmic domain generally can prevent the interaction with that domain by HIV Nef and Vpu. By preventing this interaction, these antibodies can inhibit the ability of Nef and Vpu to down-modulate CD4. Such antibodies may be directed to the cytoplasmic domain of CD4 generally, or to specific portions of the cytoplasmic domain bound by Nef or Vpu. Alternatively, antibodies might be directed to other portions of the CD4 receptor such that, when bound to those portions, the antibodies, through steric interferences or other actions, prevents or inhibits the interaction of Nef and/or Vpu with the CD4 cytoplasmic domain. Further, antibodies may be directed to the Nef, Vpu and Env gene products specifically, such that these gene products are inhibited in their ability to down-modulate CD4.

In addition to antibodies, other molecules may be provided to inhibit CD4 receptor down-modulation. Such protein molecules may bind to the cytoplasmic domain of the CD4 receptor, or to portions thereof, such that, for example, Nef and Vpu are unable to interact with the cytoplasmic domain to down-modulate CD4. Alternatively, these proteins may bind to other portions of the CD4 receptor such that, through steric interference or other mechanism, they prevent or inhibit the ability of HIV Nef and/or Vpu to interact with the CD4 cytoplasmic domain and down-modulate the CD4. Further, proteins may be used that bind to the HIV Nef, Vpu, or Env gene products such that these gene products are prevented or inhibited in their ability to down-modulate CD4. Fusion proteins may also be utilized, such proteins comprising any of various proteins fused to a portion of the CD4 receptor such that HIV gene products are unable to effectively down-modulate CD4 receptor levels.

In addition to the above, the present invention provides nucleic acids that are able to inhibit or prevent HIV down-modulation of CD4. It is known, for example, that DNA-protein interactions may be formed such that DNA is bound to protein and vice versa. These nucleic acids may act by binding to the cytoplasmic domain of CD4 such that HIV Nef and/or Vpu cannot bind thereto. By inhibiting or preventing Nef and Vpu binding of the CD4 cytoplasmic domain, the ability of HIV to down-modulate CD4 is inhibited. Alternatively, nucleic acids may be provided that bind directly to HIV nef, vpu and env gene products such that these gene products are unable to interact with the CD4 cytoplasmic domain, thereby preventing or inhibiting the ability of HIV to down-modulate CD4. Also, nucleic acids may be provided that bind to cellular proteins that interact with Nef, Vpu or other HIV gene products. These nucleic acids effectively prevent CD4 down-modulation by preventing the interaction between Nef, Vpu, or other HIV gene products and the necessary cellular proteins.

In addition to the nucleic acid molecules described above, siRNA, triplex-forming oligonucleotides, and antisense oligonucleotides may also be provided. It is contemplated that such antisense nucleic acids bind to Nef, Vpu and/or Env mRNAs, thereby inhibiting translation and formation of the nef, vpu and env gene products. As intracellular levels of Nef, Vpu, and Env are diminished, the ability of HIV to down-modulate CD4 is reduced.

Cell Fusion Assay

The present invention is also directed to a high-throughput assay for small molecule inhibitors of HIV-induced down-modulation of CD4. Fusion between gp120-expressing cells and CD4-positive cells can be modulated by the concentration of these proteins on the cell surface. Because HIV down-modulates the expression of CD4, interfering with HIV-induced down-modulation of CD4 results in higher levels of cell surface CD4, and therefore a higher rate of fusion than in cells where efficient HIV-induced CD4 down-modulation has taken place.

Although the extent of fusion may be measured by a variety of suitable methods, in various embodiments it is measured using the beta-lactamase enzyme. In these embodiments, beta-lactamase is provided via a modified HIV virus. The enzyme is cloned into the env or vpu region of an envelope-defective HIV construct. Under such conditions, expression of beta-lacatamase requires productive HIV infection and expression of early and late viral genes. This reduces the detection of false positive signals that might appear in the presence of, for example, inhibitors of HIV expression (e.g., HIV transcription, export of viral mRNAs, etc.) in systems wherein beta-lactamase is delivered into cells immediately subsequent to infection. Using the present invention, such inhibitors would block beta-lactamase expression and result in negative results. Similarly, inhibitors of HIV entry would block the transfer of beta-lactamase to substrate-loaded cells. Therefore, these unwanted events are not detected as positives by the assay of the present invention. Occurrences of false positives may be further minimized by delaying the addition of potential inhibitors several hours post-infection. This further precludes detection of inhibitors that interfere with early events (i.e., entry, reverse transcription, nuclear transport, integration, etc.). Another advantage of the present assay is that it has a built-in toxicity screen. Drugs inducing cellular toxicity may cause cell death before fusion or prevent beta-lactamase expression in infected cells, and thus such drugs will not lead to positive results in the present assay. An exemplary assay utilizing these features is further described in Example 7 below.

Competitive Inhibition

Another aspect of the present invention relies on competitive inhibition in order to inhibit HIV-induced CD4 down-modulation. In this aspect of the present invention compounds that compete with cellular proteins for binding of HIV gene products are introduced into an HIV-infected cell. In one embodiment of the present invention, CD4 cytoplasmic domain may be provided to an infected mammal by, for example, introduction of the cytoplasmic domain into the bloodstream. In this embodiment, the cytoplasmic domain may be provided with a protein transduction domain or other tag in order to allow the cytoplasmic domain to gain entry into the infected cells. Once a plurality of cytoplasmic domains enter the infected cells, they will compete with endogenous CD4 receptor cytoplasmic domain in binding the compounds necessary for HIV-induced CD4 down-modulation.

Alternatively, a CD4 cytoplasmic domain may be delivered to an infected cell via an appropriate vector. In this embodiment of the present invention, the vector contains a nucleotide sequence encoding at least a portion of the CD4 cytoplasmic domain. Once an infected cell is transduced by the vector, expression of the CD4 cytoplasmic domain can be placed under the control of a strong promoter such that excess CD4 cytoplasmic domain will be produced. The CD4 cytoplasmic domain produced by the vector will compete with endogenous CD4 receptor cytoplasmic domain in binding the compounds necessary for HIV-induced down-modulation of CD4. Any sequence encoding a CD4 cytoplasmic domain may be incorporated into a vector along with an internal ribosome entry site (IRES) such that the cytoplasmic domain and another protein may be produced from the same mRNA.

Other Therapeutics

For therapeutics, an animal, preferably a human, infected with HIV can be treated by inhibiting the ability of the HIV virus within the individual to down-modulate CD4 cell surface receptors. This can be accomplished by providing to the infected animal a vector containing DNA encoding a modified CD4 molecule that is not effectively down-modulated by HIV, by administering antisense nucleic acids that effectively inhibit HIV down-modulation of CD4, or by administering, e.g., ribozymes, oligonucleotides, siRNAs, antibodies, and other compounds that effectively inhibit the ability of HIV to down-modulate CD4 receptors.

In various embodiments, expression of therapeutic, modified CD4 molecules is confined to HIV-infected cells. Thus, uninfected cells will not be transduced with a modified CD4 receptor. This is desirable because CD4 plays an important role in the immune system, and the cytoplasmic domain of CD4 is necessary to transduce signals required for proper CD4 function. Infected cells may be targeted directly with the therapeutic CD4 molecule, or cells may be transduced with vectors that express the therapeutic CD4 molecule only in HIV-infected cells. The latter may be accomplished by placing expression of the modified CD4 molecule under the control of an HIV promoter, or under the control of a hybrid promoter that is enhanced in the presence of, for example, HIV Tat protein.

For example, in one non-limiting example of the present invention, the ability of HIV to down-modulate CD4 receptor is inhibited by administering to the infected animal a lentiviral vector, such as, for example, PPT-PGK-CD4Δcyt, pHR′-CMV-CD4Δcyt-WPRE, or pWP1-CD4Δcyt, carrying a modified CD4 molecule having a least a partial deletion of its cytoplasmic domain. When the nucleic acid encoding the modified CD4 molecule is expressed within the cells of the infected animal, the resulting CD4 receptors are less susceptible to down-modulation by HIV than their wild-type counterparts. Of course, any suitable vector may be used in such an embodiment of the present invention.

Antisense nucleic acids, ribozymes, oligonucleotides, triplex-forming oligonucleotides, siRNAs, antibodies, polypeptides, glycans, protein-methylation compounds, and other compositions, or combinations thereof, identified herein can be utilized in pharmaceutical preparations according to the Pharmaceutical Preparations and Methods of Administration section below. In various embodiments of the present invention, these various compounds act by binding to or interacting with a cytoplasmic domain of a CD4 receptor. Such binding or interaction inhibits the ability of HIV gene products to interact with a cytoplasmic domain of the CD4 receptor, thereby inhibiting the ability of HIV to down-modulate CD4.

Biological Methods

Methods involving conventional molecular biology techniques are generally known in the art and are described in detail in methodology treatises such as MOLECULAR CLONING: A LABORATORY MANUAL, 2nd ed., vol. 1-3, ed. Sambrook et al., Cold Spring Harbor Laboratory Press, Cold Spring Harbor, N.Y., 1989; and CURRENT PROTOCOLS IN MOLECULAR BIOLOGY, ed. Ausubel et al., Greene Publishing and Wiley-Interscience, New York, 1992 (with periodic updates). Various techniques using polymerase chain reaction (PCR) are described, e.g., in Innis et al., PCR PROTOCOLS: A GUIDE TO METHODS AND APPLICATIONS, Academic Press: San Diego, 1990. PCR-primer pairs can be derived from known sequences by known techniques such as using computer programs intended for that purpose. The Reverse Transcriptase Polymerase Chain Reaction (RT-PCR) method used to identify and amplify certain polynucleotide sequences within the invention may be performed as described in Elek et al., In vivo, 14:172-182, 2000. Methods and apparatus for chemical synthesis of nucleic acids are provided in several commercial embodiments, e.g., those provided by Applied Biosystems, Foster City, Calif., and Sigma-Genosys, The Woodlands, Tex. Immunological methods (e.g., preparation of antigen-specific antibodies, immunoprecipitation, and immunoblotting) are described, e.g., in CURRENT PROTOCOLS IN IMMUNOLOGY, ed. Coligan et al., John Wiley & Sons, New York, 1991; and METHODS OF IMMUNOLOGICAL ANALYSIS, ed. Masseyeff et al., John Wiley & Sons, New York, 1992. Conventional methods of gene transfer and gene therapy can also be adapted for use in the present invention. See, e.g., GENE THERAPY: PRINCIPLES AND APPLICATIONS, ed. T. Blackenstein, Springer Verlag, 1999; GENE THERAPY PROTOCOLS (METHODS IN MOLECULAR MEDICINE), ed. P. D. Robbins, Humana Press, 1997; and RETRO-VECTORS FOR HUMAN GENE THERAPY, ed. C. P. Hodgson, Springer Verlag, 1996.

Antibodies

CD4 receptor cytoplasmic domain, fragments, or variants thereof, can be used to raise antibodies useful in the invention (namely, antibodies that interfere with HIV-induced CD4 down-modulation). In general, CD4 protein, fragments of proteins, or variants thereof can be coupled to a carrier protein, such as keyhole limpet hemocyanin (KLH) or transferrin, mixed with an adjuvant, and injected into a host mammal. Antibodies produced in that animal can then be purified by peptide antigen affinity chromatography. In particular, various host animals can be immunized by an injection with CD4 receptor protein or antigenic fragments thereof. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, transferrin, and dinitrophenol.

Polyclonal antibodies are heterogeneous populations of antibody molecules that are contained in the sera of the immunized animals. Antibodies intended to be within the scope of the present invention, therefore, include polyclonal antibodies and monoclonal antibodies, single chain antibodies, Fab fragments, F(ab′)₂ fragments, and molecules produced using a Fab expression library. Monoclonal antibodies, which are homogeneous populations of antibodies to a particular antigen, can be prepared using the CD4 receptor protein described above, or a fragment thereof, such as, for example, a cytoplasmic domain thereof, and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., MONOCLONAL ANTIBODIES AND T CELL HYBRIDOMAS, Elsevier, N.Y., 1981; Ausubel et al., supra). In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., MONOCLONAL ANTIBODIES AND CANCER THERAPY, Alan R. Liss, Inc., pp. 77-96, 1983). Such antibodies can be of any immunoglobulin class including IgG, IgM, IgE, IgA, IgD and any subclass thereof. A hybridoma producing a mAb of the invention may be cultivated in vitro or in vivo. The ability to produce high titers of mAbs in vivo makes this a particularly useful method of production.

Once produced, polyclonal or monoclonal antibodies can be tested for specific recognition of, for example, the cytoplasmic domain of the CD4 cell surface receptor, by Western blot or immunoprecipitation analysis by standard methods. Antibodies that specifically bind to portions of the CD4 receptor, including the CD4 cytoplasmic domain, are useful in the invention. For example, such antibodies can be used to bind to portions of the CD4 receptor, such as the cytoplasmic domain, that are necessary to the ability of HIV to down-modulate CD4. These antibodies can be used therapeutically, being administered in a suitable manner to a mammal having HIV, or suspected of having HIV. Once successfully delivered, these antibodies are able to bind to the cytoplasmic domain of CD4 necessary to the ability of HIV to down-modulate CD4. Additionally, antibodies that bind to HIV gene products involved in CD4 down-modulation are also useful in connection with the present invention. By binding to these gene products, antibodies interfere with the function or functions of these gene products. Likewise, antibodies directed toward cellular proteins involved in HIV-induced down-modulation of CD4 may also inhibit the down-modulation of CD4 by preventing the cellular proteins from interacting with HIV gene products.

Proteins that Associate with CD4

The invention also features methods for identifying polypeptides that can associate with a CD4 receptor, e.g., the cytoplasmic domain of the CD4 receptor, or with Nef or Vpu. Any method that is suitable for detecting protein-protein interactions may be used to detect polypeptides that associate with CD4. Examples of such methods include co-immunoprecipitation, crosslinking, and co-purification through gradients or chromatographic columns or cell lysates or proteins obtained from cell lysates and the use of a CD4 protein to identify proteins in the lysate that interact with CD4 protein. For these assays, a complete CD4 protein, or some fragment thereof may be used. Once a polypeptide that associates with some portion of a CD4 protein is identified, the interacting polypeptide can be characterized, cloned, and used, in conjunction with standard techniques, to alter the activity of the protein with which it interacts. For example, such a polypeptide may be used to alter the susceptibility of CD4 to down-modulation by HIV. As with antibodies, as described above, administering such proteins to a HIV-infected mammal can inhibit the ability of HIV to down-modulate CD4 within the infected mammal. Thus, the use of such proteins has a therapeutic effect in an infected mammal.

Additionally, methods can be employed that result in the identification of genes that encode products that interact with CD4. These methods include, for example, screening expression libraries in a manner similar to the well-known technique of antibody probing of lgt₁₁ libraries, using labeled CD4 protein, CD4 fusion proteins, or fragments of CD4 proteins, fused to a marker such as an enzyme, fluorescent dye, luminescent protein, IgF_{c} domain, or other suitable marker.

Screening for Compounds that Interact with CD4 Receptor

The invention also encompasses methods for identifying compounds that specifically bind to at least a portion of a CD4 receptor, e.g., the cytoplasmic domain of the CD4 receptor. One such method, for example, involves the steps of providing immobilized purified CD4 receptor, or a fragment thereof, and a test compound; contacting the immobilized protein with the test compound; washing away substances not bound to the immobilized protein; and detecting whether or not the test compound is bound to the immobilized protein. Those compounds remaining bound to the immobilized protein are those that specifically interact with at least a portion of the CD4 receptor, or a fragment thereof. In various embodiments of the present invention, such compounds are screened for interaction with a cytoplasmic domain of CD4. Those compounds identified as having such an interaction can be used therapeutically to inhibit the ability of HIV to down-modulate CD4 in an infected mammal.

Use of CD4 Cytoplasmic Domain-Specific Antibodies

Antibodies of this invention can be used as inhibitors of CD4 cytoplasmic domain function and expression. These same antibodies can also be used to assay the expression levels of CD4 receptor having an intact cytoplasmic domain. Antibodies raised to CD4 generally may also be used to assay total CD4 levels.

Standard methods using antibodies can be used to detect and quantitate CD4 expression, including but not limited to: radioimmunoassays (“RIA”), receptor assays, enzyme immunoassays (“EIA”), cytochemical bioassays, ligand assays, immunoradiometric assays, fluoroimmunoassays, and enzyme-linked immunosorbent assays (“ELISA”). These methods are well known and will be understood by those skilled in the art to require a reasonable amount of experimentation to optimize the interaction between antibodies and antigens and the detection of the antigens by the antibodies. These and other immunoassay techniques may be found in PRINCIPLES AND PRACTICE OF IMMUNOASSAY, 2nd Ed., Price and Newman, eds., MacMillan (1997); and ANTIBODIES: A LABORATORY MANUAL, Harlow and Lane, eds., Cold Spring Harbor Laboratory, Ch. 9 (1988), each of which is incorporated herein by reference in its entirety.

The use of antibodies described herein can be used to screen samples of, e.g., peripheral blood mononuclear cells (“PBMC”) for the presence of CD4 receptor. Obtaining samples of PBMC from human or animal subjects is a well-known technique that can be practiced by anyone skilled in the art. The antibodies used in such methods are preferably monoclonal antibodies.

In various embodiments, for ease of detection and because of its quantitative nature, is the sandwich or double antibody assay of which a number of variations exist, all of which are intended to be encompassed by the present invention. For example, in a typical forward sandwich assay, unlabeled antibody is immobilized on a solid substrate, e.g., microtiter plate wells, and the sample to be tested is brought into contact with the bound molecule. After incubation for a period of time sufficient to allow formation of an antibody-antigen binary complex, a second antibody labeled with a reporter molecule capable of inducing a detectable signal is then added. Incubation is continued, allowing sufficient time for binding with the antigen at a different site and the formation of a ternary complex of antibody-antigen-labeled antibody. Any unreacted material is washed away, and the presence of the antigen is determined by observation of a signal which may be quantitated by comparison with a control sample containing known amounts of antigen. Variations on the forward sandwich assay include the simultaneous assay in which both sample and antibody are added simultaneously to the bound antibody, or a reverse sandwich assay in which the labeled antibody and sample to be tested are first combined, incubated and added to the unlabelled surface bound antibody. These techniques are well known to those skilled in the art, and the possibility of minor variations will be readily apparent. As used herein, the term “sandwich assay” is intended to encompass all variations on the basic two-site technique.

A number of combinations are possible in the identity and the way that antibodies are used for sandwich assays. As a more specific example, in a typical forward sandwich assay, a primary antibody is either covalently or non-covalently bound to a solid support. The solid surface is usually glass or a polymer, the most commonly used polymers being cellulose, polyacrylamide, nylon, polystyrene, polyvinylchloride or polypropylene. The solid supports may be in the form of tubes, beads, discs or microplates, or any other surfaces suitable for conducting an immunoassay. The binding processes are well known in the art.

Following binding, the solid phase-antibody complex is washed in preparation for the test sample. An aliquot of the sample to be tested is then added to the solid phase complex and incubated at about 25° C. for a period of time sufficient to allow binding of any CD4 protein present to the antibody specific for a particular epitope, fragment or variant. The second antibody is then added to the solid phase complex and incubated at about 25° C. for an additional period of time sufficient to allow the second antibody to bind to the primary antibody-antigen solid phase complex. The second antibody is linked to a reporter molecule, the visible signal of which is used to indicate the binding of the second antibody to any antigen in the sample. The term “reporter molecule” as used in the present invention is meant a molecule which by its chemical nature provides an analytically detectable signal which allows the detection of antigen-bound antibody. Detection must be at least relatively quantifiable to allow determination of the amount of antigen in the sample. The signal may be calculated in absolute terms or may be calculated in comparison with a standard (or series of standards) containing a known normal level of antigen.

The most commonly used reporter molecules of this type of assay are either enzymes or fluorophores. In the case of an EIA, an enzyme is conjugated to the second antibody, often by means of glutaraldehyde or periodate. As will be apparent to those skilled in the art, a wide variety of different conjugation techniques exist. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates which yield a fluorescent product rather than the chromogenic substrates noted above. In all cases, the enzyme-labeled antibody is added to the first antibody-synthesis marker or antibody-degradation marker complex and allowed to bind to the complex, then the excess reagent is washed away. A solution containing the appropriate substrate is then added to the tertiary complex of antibody-antigen-labeled antibody. The substrate reacts with the enzyme linked to the second antibody, giving a qualitative visual signal, which may be further quantitated, usually spectrophotometrically, to give an evaluation of the amount of antigen which is present in the biological fluid, tissue or organ sample.

Alternatively, fluorescent compounds such as fluorescein or rhodamine may be chemically coupled to antibodies without altering their binding capacity. When activated by illumination with light of a particular wavelength, the fluorochrome-labeled antibody absorbs the light energy inducing a state of excitability in the molecule followed by emission of the light at a longer wavelength. The emission appears as a characteristic color visually detectable with a light microscope. As in the EIA, the fluorescent-labeled antibody is allowed to bind to the first antibody-synthesis marker or antibody-degradation marker complex. After washing the unbound reagent, the remaining ternary complex is then exposed to light of the appropriate wavelength, and the fluorescence observed indicates the presence of the antigen. Other reporter molecules, such as radioisotopes, chemiluminescent or bioluminescent molecules may also be employed in the assays of the present invention. It will be readily apparent to those skilled in the art how to vary the procedure to suit the required use.

In yet another alternative embodiment, the sample to be tested which contains the CD4 receptor, e.g., the cytoplasmic domain of the CD4 receptor, may be used in a single site immunoassay wherein it is adhered to a solid substrate either covalently or noncovalently. An unlabeled anti-CD4 antibody is brought into contact with the sample bound on the solid substrate. After a suitable period of incubation sufficient to allow formation of an antibody-CD4 binary complex, a second antibody, labeled with a reporter molecule capable of inducing a detectable signal is added and incubation is continued allowing sufficient time for the formation of a ternary complex of antigen-antibody-labeled antibody. For the single site immunoassay, the second antibody may be a general antibody, i.e., zenogeneic antibody to immunoglobulin, particularly anti-(IgM and IgG) linked to a reporter molecule, capable of binding an antibody that is specific for the CD4 receptor or, particularly, the cytoplasmic domain thereof.

Immunofluorescent histological techniques can also be used to examine human or other mammalian specimens with monoclonal antibodies. In a typical protocol, slides containing cryostat sections of frozen, unfixed tissue biopsy samples or cytological smears are air dried, formalin-fixed and incubated with the monoclonal antibody preparation in a humidified chamber at room temperature. The slides are then layered with a preparation of antibody directed against the monoclonal antibody, usually an anti-mouse immunoglobulin if the monoclonal antibodies used are derived from the fusion of a mouse spleen lymphocyte and a mouse myeloma cell line. This antimouse immunoglobulin is tagged with a compound that fluoresces at a particular wavelength, e.g., rhodamine or fluorescein isothiocyanate. The staining pattern and intensities within the sample are then determined by fluorescent light microscopy and optionally photographically recorded.

Monoclonal antibodies which can be used in the invention can be produced by a hybridoma using methods well known in the art. Various additional procedures known in the art may be used for the production of antibodies to epitopes of particular portions of the CD4 receptor, e.g., the cytoplasmic domain of the CD4 receptor. Such antibodies include but are not limited to polyclonal, monoclonal, chimeric, single chain, Fab fragments and a Fab expression library. The production of antibodies may be accomplishes as described above.

Solid Phase Assays

The solid phase used in the assays of this invention may be any surface commonly used in immunoassays. For example, the solid phase may be particulate; it may be the surface of beads, e.g., glass or polystyrene beads, or it may be the solid wall surface of any of a variety of containers, e.g., centrifuge tubes, columns, microtiter plate wells, filters, membranes and tubing, among other containers.

When particles are used as the solid phase, they may be of a size in the range of from about 0.4 to 200 microns, particularly from about 0.8 to 4.0 microns. In various embodiments, magnetic or magnetizable particles, such as paramagnetic particles (PMP), and microtiter plate wells as a solid wall surface are particularly useful. Magnetic or magnetizable particles may be particularly useful when the steps of the methods of this invention are performed in an automated immunoassay system.

In various embodiments, detection/quantitation systems of this invention may be luminescent, and a luminescent detection/quantitation system in conjunction with a signal amplification system could be used, if necessary. Exemplary luminescent labels, e.g., chemiluminescent labels, are detailed below, as are signal amplification systems.

Signal Detection/Quantitation Systems

The complexes formed by the assays of this invention can be detected, or detected and quantitated by any known detection/quantitation systems used in immunoassays. As appropriate, the antibodies of this invention used as tracers may be labeled in any manner directly or indirectly, that results in a signal that is visible or can be rendered visible.

Detectable marker substances include radionuclides, such as ³H, ¹²⁵I, and ¹³¹I; fluorescers, such as, fluorescein isothiocyanate and other fluorochromes, phycobiliproteins, phycoerythin, rare earth chelates, Texas red, dansyl and rhodamine; colorimetric reagents (chromogens); electron-opaque materials, such as colloidal gold; bioluminescers; chemiluminescers; dyes; enzymes, such as, horseradish peroxidase, alkaline phosphatase, glucose oxidase, glucose-6-phosphate dehydrogenase, acetylcholinesterase, α-, beta-galactosidase, among others; coenzymes; enzyme substrates; enzyme cofactors; enzyme inhibitors; enzyme subunits; metal ions; free radicals; or any other immunologically active or inert substance which provides a means of detecting or measuring the presence or amount of immunocomplex formed. Exemplary of enzyme substrate combinations are horseradish peroxidase and tetramethyl benzidine (TMB), and alkaline phosphatase and paranitrophenyl phosphate (pNPP).

In various embodiment, the detection or detection and quantitation systems according to this invention, produce luminescent signals, bioluminescent (BL) or chemiluminescent (CL). In CL or BL assays, the intensity or the total light emission is measured and related to the concentration of the analyte. Light can be measured quantitatively using a luminometer (photomultiplier tube as the detector) or charge-coupled device, or qualitatively by means of photographic or X-ray film. The main advantages of using such assays is their simplicity and analytical sensitivity, enabling the detection and/or quantitation of very small amounts of analyte.

Exemplary luminescent labels are acridinium esters, acridinium sulfonyl carboxamides, luminol, umbelliferone, isoluminol derivatives, photoproteins, such as aequorin, and luciferases from fireflies, marine bacteria, Vargulla and Renilla. Luminol can be used optionally with an enhancer molecule, in particular 4-iodophenol or 4-hydroxycinnamic acid. A signal is generated by treatment with an oxidant under basic conditions.

Luminescent detection systems also include those wherein the signal is produced by an enzymatic reaction upon a substrate. CL and BL detection schemes have been developed for assaying alkaline phosphatase (AP), glucose oxidase, glucose 6-phosphate dehydrogenase, horseradish peroxidase (HRP), and xanthine-oxidase labels, among others. AP and HRP are two particular enzyme labels which can be quantitated by a range of CL and BL reactions. For example, AP can be used with a substrate, such as an adamantyl 1,2-dioxetane aryl phosphate substrate (e.g., AMPPD or CSPD; (Kricka, L. J., “Analysis by Chemiluminescence and Bioluminescence” at p. 167, MOLECULAR BIOLOGY AND BIOTECHNOLOGY: A COMPREHENSIVE DESK REFERENCE, ed. R. A. Meyers, VCH Publishers; N.Y., N.Y.; 1995)); a disodium salt of 4-methoxy-4-(3-phosphatephenyl)spiro[1,2-d]oxetane-3,2′-adamantane], with or without an enhancer molecule, such as 1-(trioctylphosphonium methyl)-4-(tributylphosphonium methyl) benzene diochloride. HRP may be used with substrates, such as, 2′,3′,6′-trifluorophenyl 3-methoxy-10-methylacridan-9-carboxylate.

CL and BL reactions can also be adapted for analysis of not only enzymes, but other substrates, cofactors, inhibitors, metal ions and the like. For example, luminol, firefly luciferase, and marine bacterial luciferase reactions are indicator reactions for the production or consumption of peroxide, ATP, and NADPH, respectively. They can be coupled to other reactions involving oxidases, kinases, and dehydrogenases, and can be used to measure any component of the coupled reaction (enzyme, substrate, cofactor).

The detectable marker may be directly or indirectly linked to an antibody used in an assay of this invention. Exemplary of an indirect linkage of the detectable label is the use of a binding pair between the antibody and the marker, or the use of well known signal amplification signals, such as, using a biotinylated antibody complexed to UGP and then adding streptavidin conjugated to HRP and then TMB.

Exemplary of binding pairs that can be used to link antibodies of assays of this invention to detectable markers are biotin/avidin, streptavidin, or anti-biotin; avidin/anti-avidin; thyroxine/thyroxine-binding globulin; antigen/antibody; antibody/anti-antibody; carbohydrate/lectins; hapten/anti-hapten antibody; dyes and hydrophobic molecules/hydrophobic protein binding sites; enzyme inhibitor, coenzyme or cofactor/enzyme; polynucleic acid/homologous polynucleic acid sequence; fluorescein/anti-fluorescein; dinitrophenol/anti-dinitrophenol; vitamin B12/intrinsic factor; cortisone, cortisol/cortisol binding protein; and ligands for specific receptor protein/membrane associated specific receptor proteins. Particular binding pairs according to this invention are biotin/avidin or streptavidin, more particularly biotin/streptavidin.

Various means for linking labels directly or indirectly to antibodies are known in the art. For example, labels may be bound either covalently or non-covalently. Exemplary antibody conjugation methods are described in: Avarmeas, et al., Scan. J. Immunol., 8 (Suppl. 7):7 (1978); Bayer, et al., Meth. Enzymol., 62:308 (1979); Chandler, et al., J. Immunol. Meth., 53:187 (1982); Ekeke and Abuknesha, J. Steroid Biochem., 11:1579 (1979); Engvall and Perlmann, J. Immunol., 109:129 (1972); Geoghegan, et al., Immunol. Comm., 7:1 (1978); and Wilson and Nakane, Immunofluorescence and Related Techniques, p. 215 (Elsevier/North Holland Biomedical Press; Amsterdam (1978)).

Depending upon the nature of the label, various techniques can be employed for detecting, or detecting and quantitating the label. For fluorescers, a large number of fluorometers are available. For chemiluminescers, luminometers or films are available. With enzymes, a fluorescent, chemiluminescent, or colored product can be determined or measured fluorometrically, luminometrically, spectrophotometrically or visually.

Automated Immunoassay Systems

The methods of this invention can be readily adapted to automated immunochemistry analyzers. To facilitate automation of the methods of this invention and to reduce the turnaround time, anti-UGP antibodies may be coupled to magnetizable particles.

Exemplary automated/immunoassay systems include the DPC Immulite® system (Los Angeles, Calif. (USA)), Advia, IMS (Bayer Corp., Pittsburgh, Pa. (USA)), Bayer ACS: 180TM Automated Chemiluminescence System (CCD; Medfield, Mass. (USA), Beckman Access (South San Francisco, Calif. (USA), Abbott AxSYM (Chicago, Ill. (USA)), and the like. The systems use chemiluminescent labels as tracers and paramagnetic particles as solid-phase reagents. The ACS:180 system accommodates both competitive binding and sandwich-type assays, wherein each of the steps are automated. The ACS:180 uses micron-sized paramagnetic particles that maximize the available surface area, and provide a means of rapid magnetic separation of bound tracer from unbound tracer without centrifugation. Reagents can be added simultaneously or sequentially. Other tags, such as an enzymatic tag, can be used in place of a chemiluminescent label, such as, acridinium ester.

Nucleic Acids Encoding CD4 Proteins

Various nucleic acid molecules may be used in the invention which modified CD4 receptor polynucleotides, e.g., the polynucleotide having a nucleotide sequence of SEQ ID NO:2. Another nucleic acid that can be used in various aspects of the invention includes a purified nucleic acid or polynucleotide that encodes a polypeptide having the amino acid sequences of SEQ ID NOs:1 and 3.

Nucleic acid molecules utilized in the present invention may be in the form of RNA or in the form of DNA (e.g., cDNA, genomic DNA, and synthetic DNA). The DNA may be double-stranded or single-stranded and if single-stranded, may be the coding (sense) strand or non-coding (anti-sense) strand. The coding sequence that encodes a modified CD4 receptor protein may be identical to the nucleotide sequence of SEQ ID NO:2, or it may also be a different coding sequence that, as a result of the redundancy or degeneracy of the genetic code, encodes the same polypeptide as the polynucleotides of SEQ ID NO:1. Likewise, coding sequences encoding a wild-type CD4 receptor that, as a result of the redundancy or degeneracy of the genetic code, encode for the same polypeptide as the polynucleotides of SEQ ID NO:1 are also contemplated. Examples of nucleotide codons which provide the same expressed amino acid are summarized in Table 1:

TABLE 1

| | | Abbreviation | Abbreviation |
|---|---|---|---|
| Codon | Full Name | (3 Letter) | (1 Letter) |
| TTT | Phenylalanine | Phe | F |
| TTC | Phenylalanine | Phe | F |
| TTA | Leucine | Leu | L |
| TTG | Leucine | Leu | L |
| TCT | Serine | Ser | S |
| TCC | Serine | Ser | S |
| TCA | Serine | Ser | S |
| TCG | Serine | Ser | S |
| TAT | Tyrosine | Tyr | Y |
| TAC | Tyrosine | Tyr | Y |
| TAA | Termination | Ter | X |
| TAG | Termination | Ter | X |
| TGT | Cysteine | Cys | C |
| TGC | Cysteine | Cys | C |
| TGA | Termination | Ter | X |
| TGG | Tryptophan | Trp | W |
| CTT | Leucine | Leu | L |
| CTC | Leucine | Leu | L |
| CTA | Leucine | Leu | L |
| CTG | Leucine | Leu | L |
| CCT | Proline | Pro | P |
| CCC | Proline | Pro | P |
| CCA | Proline | Pro | P |
| CCG | Proline | Pro | P |
| CAT | Histidine | His | H |
| CAC | Histidine | His | H |
| CAA | Glutamine | Gln | Q |
| CAG | Glutamine | Gln | Q |
| CGT | Arginine | Arg | R |
| CGC | Arginine | Arg | R |
| CGA | Arginine | Arg | R |
| CGG | Arginine | Arg | R |
| ATT | Isoleucine | Ile | I |
| ATC | Isoleucine | Ile | I |
| ATA | Isoleucine | Ile | I |
| ATG | Methionine | Met | M |
| ACT | Threonine | Thr | T |
| ACC | Threonine | Thr | T |
| ACA | Threonine | Thr | T |
| ACG | Threonine | Thr | T |
| AAT | Asparagine | Asn | N |
| AAC | Asparagine | Asn | N |
| AAA | Lysine | Lys | K |
| AAG | Lysine | Lys | K |
| AGT | Serine | Ser | S |
| AGC | Serine | Ser | S |
| AGA | Arginine | Arg | R |
| AGG | Arginine | Arg | R |
| GTT | Valine | Val | V |
| GTC | Valine | Val | V |
| GTA | Valine | Val | V |
| GTG | Valine | Val | V |
| GCT | Alanine | Ala | A |
| GCC | Alanine | Ala | A |
| GCA | Alanine | Ala | A |
| GCG | Alanine | Ala | A |
| GAT | Aspartate | Asp | D |
| GAC | Aspartate | Asp | D |
| GAA | Glutamate | Glu | E |
| GAG | Glutamate | Glu | E |
| GGT | Glycine | Gly | G |
| GGC | Glycine | Gly | G |
| GGA | Glycine | Gly | G |
| GGG | Glycine | Gly | G |

Other nucleic acid molecules intended to be within the scope of the present invention include variants of the modified CD4 gene such as those that encode fragments, analogs and derivatives of a modified CD4 receptor protein. Such variants may be, e.g., naturally occurring allelic variants of the wild-type CD4 gene, homologs of the wild-type CD4 gene having mutations in a cytoplasmic domain thereof, or non-naturally occurring variants of the modified CD4 gene. These variants have nucleotide sequences that differs from the corresponding wild-type or modified CD4 genes in one or more bases. For example, the nucleotide sequences of such variants can feature deletions, additions, or substitutions of one or more nucleotides of the wild-type or modified CD4 gene. Nucleic acid insertions are preferably of about 1 to 10 contiguous nucleotides, and deletions are preferably of about 1 to 30 contiguous nucleotides. In addition, nucleic acids may be used that code for modified CD4 proteins in which the cytoplasmic domain of CD4 is replaced by a heterologous protein. This may enhance the ability of the modified CD4 protein to block HIV-induced CD down-modulation, thereby inhibiting HIV infectivity.

In other applications, variant CD4 receptor proteins displaying substantial changes in structure can be generated by making nucleotide substitutions that cause less than conservative changes in the encoded polypeptide. Examples of such nucleotide substitutions, as shown in Table 1, are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamine or aspartine; or (d) a residue having a bulky side chain, e.g., phenylalanine, for (or by) one not having a side chain, e.g., glycine. Table 2 provides similar possible substitution possibilities:

TABLE 2

| | 3- | 1- | |
|---|---|---|---|
| Amino Acid | letter code | letter code | Properties |
| Alanine | Ala | A | Aliphatic, hydrophobic, neutral |
| Arginine | Arg | R | polar, hydrophilic, charged (+) |
| Asparagine | Asn | N | polar, hydrophilic, neutral |
| Aspartate | Asp | D | polar, hydrophilic, charged (−) |
| Cysteine | Cys | C | polar, hydrophobic, neutral |
| Glutamine | Gln | Q | polar, hydrophilic, neutral |
| Glutamate | Glu | E | polar, hydrophilic, charged (−) |
| Glycine | Gly | G | aliphatic, neutral |
| Histidine | His | H | aromatic, polar, hydrophilic, |
| | | | charged (+) |
| Isoleucine | Ile | I | aliphatic, hydrophobic, neutral |
| Leucine | Leu | L | aliphatic, hydrophobic, neutral |
| Lysine | Lys | K | polar, hydrophilic, charged (+) |
| Methionine | Met | M | hydrophobic, neutral |
| Phenylalanine | Phe | F | aromatic, hydrophobic, neutral |
| Proline | Pro | P | hydrophobic, neutral |
| Serine | Ser | S | polar, hydrophilic, neutral |
| Threonine | Thr | T | polar, hydrophilic, neutral |
| Tryptophan | Trp | W | aromatic, hydrophobic, neutral |
| Tyrosine | Tyr | Y | aromatic, polar, hydrophobic |
| Valine | Val | V | aliphatic, hydrophobic, neutral |

Naturally occurring allelic variants of a wild-type CD4 gene or wild-type CD4 mRNAs within the invention are nucleic acids isolated from human tissue that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the wild-type CD4 gene or wild-type CD4 mRNAs, and encode polypeptides having structural similarity to a wild-type CD4 protein. Homologs of the wild-type CD4 gene or wild-type CD4 mRNAs within the invention are nucleic acids isolated from other species that have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the wild-type CD4 gene or wild-type CD4 mRNAs, and encode polypeptides having structural similarity to wild-type CD4 protein. Public and/or proprietary nucleic acid databases can be searched to identify other nucleic acid molecules having a high percent (e.g., 75, 85, 95% or more) sequence identity to the wild-type CD4 gene or wild-type CD4 mRNAs.

Non-naturally occurring wild-type CD4 gene or mRNA variants are nucleic acids that do not occur in nature (e.g., are made by the hand of man), have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the wild-type CD4 gene or wild-type CD4 mRNAs, and encode polypeptides having structural similarity to wild-type CD4 protein. Examples of non-naturally occurring wild-type CD4 gene variants are those that encode a fragment of a wild-type CD4 protein, those that hybridize to the wild-type CD4 gene or a complement of the wild-type CD4 gene under stringent conditions, those that share at least 75% sequence identity with the wild-type CD4 gene or a complement thereof, and those that encode a wild-type CD4 protein.

Naturally occurring allelic variants of a modified CD4 gene or modified CD4 mRNAs (such as those genes or mRNAs encoding CD4Δcyt) within the invention are nucleic acids isolated from human tissue that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the modified CD4 gene or modified CD4 mRNAs, and encode polypeptides having structural similarity to a modified CD4 protein. Homologs of the modified CD4 gene or modified CD4 mRNAs within the invention are nucleic acids isolated from other species that have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the modified CD4 gene or modified CD4 mRNAs, and encode polypeptides having structural similarity to modified CD4 protein. Public and/or proprietary nucleic acid databases can be searched to identify other nucleic acid molecules having a high percent (e.g., 75, 85, 95% or more) sequence identity to the modified CD4 gene or modified CD4 mRNAs.

Non-naturally occurring modified CD4 gene or mRNA variants are nucleic acids that do not occur in nature (e.g., are made by the hand of man), have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the modified CD4 gene or modified CD4 mRNAs, and encode polypeptides having structural similarity to modified CD4 protein. Examples of non-naturally occurring modified CD4 gene variants are those that encode a fragment of a modified CD4 protein, those that hybridize to the modified CD4 gene or a complement of the modified CD4 gene under stringent conditions, those that share at least 75% sequence identity with the modified CD4 gene or a complement thereof, and those that encode a modified CD4 protein.

Nucleic acids encoding fragments of a wild-type CD4 protein within the invention are those that encode, e.g., 2, 3, 4, 5, 10, 25, 50, 100, 150, 200, 250, 300, or more amino acid residues of the wild-type CD4 protein. Shorter oligonucleotides (e.g., those of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 100, 125, 150 or 200 base pairs in length) that encode or hybridize with nucleic acids that encode fragments of a wild-type CD4 protein can be used as probes, primers, or antisense molecules. Longer polynucleotides (e.g., those of 300, 400, 500, 600, 700, 800, 900, 1000, 1100, 1200, 1300, 1400, 1500, 1600, 1700, 1800, 1900 or 2000 base pairs) that encode or hybridize with nucleic acids that encode fragments of a wild-type CD4 protein can also be used in various aspects of the invention. Nucleic acids encoding fragments of a wild-type CD4 protein can be made by enzymatic digestion (e.g., using a restriction enzyme) or chemical degradation of the full length wild-type CD4 gene, a wild-type CD4 mRNA or cDNA, or variants of the foregoing.

Nucleic acids encoding fragments of a modified CD4 protein within the invention are those that encode, e.g., 2, 3, 4, 5, 10, 25, 50, 100, 150, 200, 250, 300, or more amino acid residues of the modified CD4 protein. Shorter oligonucleotides (e.g., those of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 100, 125, 150 or 200 base pairs in length) that encode or hybridize with nucleic acids that encode fragments of a modified CD4 protein can be used as probes, primers, or antisense molecules. Longer polynucleotides (e.g., those of 300, 400, 500, 600, 700, 800, 900, 1000, 1100, 1200, 1300, 1400, 1500, 1600, 1700, 1800, 1900 or 2000 base pairs) that encode or hybridize with nucleic acids that encode fragments of a modified CD4 protein can also be used in various aspects of the invention. Nucleic acids encoding fragments of a modified CD4 protein can be made by enzymatic digestion (e.g., using a restriction enzyme) or chemical degradation of the full length modified CD4 gene, a modified CD4 mRNA or cDNA, or variants of the foregoing.

Nucleic acids that hybridize under stringent conditions to the nucleic acids of SEQ ID NOs: 2 or 4 or the complements of SEQ ID NOs: 2 or 4 can also be used in the invention. For example, such nucleic acids can be those that hybridize to SEQ ID NOs: 2 or 4 or the complement of SEQ ID NOs: 2 or 4 under low stringency conditions, moderate stringency conditions, or high stringency conditions are within the invention. In various embodiments, nucleotide acids are those having a nucleotide sequence that is the complement of all or a portion of SEQ ID NOs: 2 or 4. Other variants of the wild-type or modified CD4 receptor gene within the invention are polynucleotides that share at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity to SEQ ID NOs: 2 or 4 or the complement of SEQ ID NOs: 2 or 4. Nucleic acids that hybridize under stringent conditions to or share at least 65% sequence identity with SEQ ID NOs: 2 or 4 or the complement of SEQ ID NOs: 2 or 4 can be obtained by techniques known in the art such as by making mutations in the wild-type or modified CD4 gene, or by isolation from an organism expressing such a nucleic acid (e.g., an allelic variant).

Nucleic acid molecules encoding CD4 fusion proteins are also within the invention. Such nucleic acids can be made by preparing a construct (e.g., an expression vector) that expresses an CD4 fusion protein when introduced into a suitable host. For example, such a construct can be made by ligating a first polynucleotide encoding a CD4 protein fused in frame with a second polynucleotide encoding another protein such that expression of the construct in a suitable expression system yields a fusion protein.

The nucleic acid molecules of the invention can be modified at a base moiety, sugar moiety, or the phosphate backbone, e.g., to improve stability of the molecule, hybridization, and the like. For example the nucleic acid molecules of the invention can be conjugated to groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988), hybridization-triggered cleavage agents. (See, e.g., Krol et al. (1988) BioTechniques 6:958-976) or intercalating agents (see, e.g., Zon (1988) Pharm. Res. 5:539-549).

Modified Internucleoside Linkages (Backbones)

The nucleic acid molecules of the invention include oligonucleotides containing modified backbones or non-natural internucleoside linkages. Oligonucleotides having modified backbones include those that retain a phosphorus atom in the backbone and those that do not have a phosphorus atom in the backbone. As referred to in the art, modified oligonucleotides that do not have a phosphorus atom in their internucleoside backbone can also be considered oligonucleosides.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage, i.e., a single inverted nucleoside residue which may be abasic (the nucleobase is missing or has a hydroxyl group in place thereof). Various salts, mixed salts and free acid forms are also included.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other modified oligonucleotides, both the sugar and the internucleoside linkage (i.e., the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

Various other embodiments of the invention utilize oligonucleotides with phosphorothioate backbones and oligonucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Other oligonucleotides having morpholino backbone structures are disclosed in U.S. Pat. No. 5,034,506.

Modified oligonucleotides may also contain one or more substituted sugar moieties. Such oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly included are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides may comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. An exemplary modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. An exemplary 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

A further exemplary modification of the sugar includes Locked Nucleic Acids (LNAs) in which the 2′-hydroxyl group is linked to the 3′ or 4′ carbon atom of the sugar ring, thereby forming a bicyclic sugar moiety. The linkage may be, e.g., a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Nucleobases

Oligonucleotides may also include nucleobase modifications or substitutions. As used herein and described above, unmodified and natural nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—CC—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4-]benzoxaz-in-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in THE CONCISE ENCYCLOPEDIA OF POLYMER SCIENCE AND ENGINEERING, pages 858-859, Kroschwitz, J. T., ed. John Wiley & Sons, 1990; those disclosed by Englisch et al., ANGEWANDTE CHEMIE, INTERNATIONAL EDITION, 1991, 30, 613; and those disclosed by Sanghvi, Y. S., Chapter 15, ANTISENSE RESEARCH AND APPLICATIONS, pages 289-302, Crooke, S. T. and Lebleu, B. ed., CRC Press, 1993. Certain of these nucleobases are particularly useful for increasing the binding affinity of the compounds of the invention. These include 5-substituted pyrimidines, 6-azapyrimidines and N2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even and enhanced when combined with 2′-O-methoxyethyl sugar modifications.

Conjugates

Another modification of the nucleic acids of the present invention involves chemically linking to the nucleic acid one or more moieties or conjugates that enhance the activity, cellular distribution or cellular uptake of the nucleic acid. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of nucleic acids. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention.

Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glyc-ero-3-H-phosphonate, a polyamine or a polyethylene glycol chain, or adamantane acetic acid, a palmityl moiety, or an octadecylamine or hexylamino-carbonyl-oxycholesterol moiety. Nucleic acids of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Chimeric Compounds

It is not necessary for all positions in a given compound to be uniformly modified, and in fact more than one of the aforementioned modifications may be incorporated in a single compound or even at a single nucleoside within an oligonucleotide.

The present invention also includes antisense compounds that are chimeric compounds. “Chimeric” antisense compounds or “chimeras,” in the context of this invention, are CD4 receptor nucleic acids and proteins, in particular oligonucleotides, that contain two or more chemically distinct regions, each made up of at least one monomer unit, i.e., a nucleotide in the case of an oligonucleotide compound. Such oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. For example, RNAseH is a cellular endonuclease that cleaves the RNA strand of an RNA:DNA duplex. Activation of RNaseH, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases such as RNAseL which cleaves both cellular and viral RNA. Cleavage of the RNA target can be routinely detected by gel electrophoresis and, if necessary, associated nucleic acid hybridization techniques known in the art.

Chimeric CD4 receptor nucleic acids and proteins of the invention may be formed as composite structures of, e.g., two or more oligonucleotides, modified oligonucleotides, oligonucleosides and/or oligonucleotide mimetics as described above. Such compounds have also been referred to in the art as hybrids or gapmers.

Antisense, Ribozyme, Triplex Techniques

Another aspect of the invention relates to the use of purified antisense nucleic acids, including modified oligonucleotides, to inhibit expression of HIV genes that encode products that bind to the cytoplasmic domain of a host CD4 receptor, or to cellular proteins binding to domains in Nef or Vpu important for their CD4 down-modulation activity. Antisense nucleic acid molecules within the invention are those that specifically hybridize (e.g., bind) under cellular conditions to HIV mRNA and/or genomic DNA encoding products that are capable of down-modulating cellular CD4 receptor. The binding may be by conventional base pair complementarity, or, for example, in the case of binding to DNA duplexes, through specific interactions in the major groove of the double helix.

Antisense constructs can be delivered, for example, as an expression vector that, when transcribed in the cell, produces RNA that is complementary to at least a unique portion of the HIV-encoded mRNA that encodes a product capable of down-modulating CD4 by binding to a CD4 receptor cytoplasmic domain. Alternatively, the antisense construct can take the form of an oligonucleotide probe generated ex vivo that, when introduced into a HIV gene-product expressing cell, causes inhibition of HIV gene product expression by hybridizing with an mRNA and/or genomic sequences coding for the HIV gene products. Such oligonucleotide probes may be modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668. With respect to antisense DNA, oligodeoxyribonucleotides derived from the translation initiation site are preferred.

Antisense approaches can involve the design of oligonucleotides (either DNA or RNA) that are complementary to a CD4 receptor cytoplasmic domain, or portion thereof, mRNA or HIV mRNA. The antisense oligonucleotides will bind to cytoplasmic domain or HIV mRNA transcripts and prevent translation. In various HIV mRNA embodiments, the antisense oligonucleotides will bind to mRNA transcripts of HIV nef, vpu, or env. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex or triplex. One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. Sequences complementary to the 3′ untranslated sequences of mRNAs, however, have been shown to be effective at inhibiting translation of mRNAs as well. (Wagner, R. (1994) Nature 372:333). Therefore, oligonucleotides complementary to either the 5′ or 3′ untranslated, non-coding regions of a HIV gene could be used in an antisense approach to inhibit translation of endogenous HIV mRNA. Oligonucleotides complementary to the 5′ untranslated region of the mRNA may include the complement of the AUG start codon. Although antisense oligonucleotides complementary to mRNA coding regions are generally less efficient inhibitors of translation, these could still be used in the invention. Whether designed to hybridize to the 5′, 3′ or coding region of a CD4 receptor cytoplasmic domain mRNA or HIV mRNA, preferred antisense nucleic acids are less that about 100 (e.g., less than about 30, 25, 20, or 18) nucleotides in length. Generally, in order to be effective, the antisense oligonucleotide should be 18 or more nucleotides in length, but may be shorter depending on the conditions.

Specific antisense oligonucleotides can be tested for effectiveness using in vitro studies to assess the ability of the antisense oligonucleotide to inhibit gene expression. such studies generally (1) utilize controls (e.g., a non-antisense oligonucleotide of the same size as the antisense oligonucleotide) to distinguish between antisense gene inhibition and nonspecific biological effects of oligonucleotides, and (2) compare levels of the target RNA or protein with that of an internal control RNA or protein.

Antisense oligonucleotides of the invention may include at least one modified base or sugar moiety such as those provided above. Antisense oligonucleotides within the invention might also be an alpha-anomeric oligonucleotide. See, Gautier et al. (1987) Nucl. Acids Res. 15:6625-6641. For example, the antisense oligonucleotide can be a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Oligonucleotides of the invention may be synthesized by standard methods known in the art, e.g., by use of an automated DNA synthesizer, as described above. Phosphorothioate oligonucleotides may be synthesized by the method of Stein et al. (1988) Nucl. Acids Res. 16:3209). Methylphosphonate oligonucleotides can be prepared by use of controlled pore glass polymer supports (e.g., as described in Sarin et al. (1988) Proc. Natl. Acad. Sci. U.S.A. 85:7448-7451).

The invention also provides a method for delivering one or more of the above-described nucleic acid molecules into cells that express HIV gene products. A number of methods have been developed for delivering antisense DNA or RNA into cells. For example, antisense molecules can be introduced directly into a cell by electroporation, liposome-mediated transfection, CaCl-mediated transfection, or using a gene gun. Modified nucleic acid molecules designed to target the desired cells (e.g., antisense oligonucleotides linked to peptides or antibodies that specifically bind receptors or antigens expressed on the target cell surface) can be used. To achieve high intracellular concentrations of antisense oligonucleotides (as may be required to suppress translation on endogenous mRNAs), a preferred approach utilizes a recombinant DNA construct in which the antisense oligonucleotide is placed under the control of a strong promoter, e.g., the CMV promoter or a HIV promoter.

Ribozymes

Ribozyme molecules designed to catalytically cleave CD4 receptor cytoplasmic domain mRNA or HIV mRNA transcripts can also be used to prevent translation of CD4 receptor cytoplasmic domain HIV gene products (see, e.g., Wright and Kearney, Cancer Invest. 19:495, 2001; Lewin and Hauswirth, Trends Mol. Med. 7:221, 2001; Sarver et al. (1990) Science 247:1222-1225 and U.S. Pat. No. 5,093,246). As one example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach (1988) Nature 334:585-591. As another example, hairpin and hepatitis delta virus ribozymes may also be used. See, e.g., Bartolome et al. (2004) Minerva Med. 95(1):11-24. To increase efficiency and minimize the intracellular accumulation of non-functional mRNA transcripts, a ribozyme should be engineered so that the cleavage recognition site is located near the 5′ end of the target CD4 receptor cytoplasmic domain mRNA or HIV mRNA. Ribozymes within the invention can be delivered to a cell using a vector as described below.

Other methods can also be used to reduce CD4 receptor cytoplasmic domain mRNA or HIV gene expression in a cell. For example, CD4 receptor cytoplasmic domain or HIV gene expression can be reduced by inactivating or “knocking out” any of various CD4 receptor cytoplasmic domain or HIV genes or their promoters using targeted homologous recombination. See, e.g., Kempin et al., Nature 389: 802 (1997); Smithies et al. (1985) Nature 317:230-234; Thomas and Capecchi (1987) Cell 51:503-512; and Thompson et al. (1989) Cell 5:313-321. For example, a mutant, non-functional CD4 receptor cytoplasmic domain or HIV gene variant (or a completely unrelated DNA sequence) flanked by DNA homologous to the CD4 receptor cytoplasmic domain or HIV gene (either the coding regions or regulatory regions of the HIV gene) can be used, with or without a selectable marker and/or a negative selectable marker, to transfect cells that express CD4 receptor cytoplasmic domain or HIV gene products in vivo.

CD4 receptor cytoplasmic domain expression might also be reduced by targeting deoxyribonucleotide sequences complementary to particular regions of CD4 receptor cytoplasmic domain gene (i.e., promoters and/or enhancers of the CD4 receptor cytoplasmic domain) to form triple helical structures that prevent transcription of the gene(s) in target cells. See generally, Helene, C. (1991) Anticancer Drug Des. 6(6): 569-84; Helene, C., et al. (1992) Ann. N.Y. Acad. Sci. 660:27-36; and Maher, L. J. (1992) Bioassays 14(12): 807-15. Other gene targets useful in the present invention include HIV nef, vpu, and env genes, and other cellular genes necessary for the virus-induced down-modulation of CD4. Nucleic acid molecules to be used in this technique may be single stranded and composed of deoxyribonucleotides. The base composition of these oligonucleotides should be selected to promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art, such as for example, solid phase phosphoramide chemical synthesis. RNA molecules may be generated by in vitro and in vivo transcription of DNA sequences encoding the antisense RNA molecule. Such DNA sequences may be incorporated into a wide variety of vectors that incorporate suitable RNA polymerase promoters. Alternatively, antisense cDNA constructs that synthesize antisense RNA constitutively or inducibly, depending on the promoter used, can be introduced stably into cell lines.

RNA Interference (RNAi)

The use of short-interfering RNA (siRNA) is a technique known in the art for inhibiting expression of a target gene by introducing exogenous RNA into a living cell (Elbashir et al. 2001. Nature. 411:494-498). siRNAs suppress gene expression through a highly regulated enzyme-mediated process called RNA interference (RNAi). RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial to performing efficient RNAi. Reynolds et al. provide methods for identifying such features. See, A. Reynolds et al., Rational siRNA design for RNA interference, Nature Biotechnology 22(3), March 2004. In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high Tm values would favor internal hairpin structures. In a preferred embodiment of the present invention, such siRNA sequences are directed toward inhibiting the expression of HIV nef, vpu, or env genes, or some combination thereof, or cellular genes necessary for the virus-induced down-modulation of CD4.

Similar to other inhibitors disclosed herein, siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The extent to which there is loss of function of the target gene can be titrated using the dose of double stranded RNA delivered. A reduction or loss of gene expression in at least 99% of targeted cells has been shown. See, e.g., U.S. Pat. No. 6,506,559. Lower doses of injected material and longer times after administration of siRNA may result in inhibition in a smaller fraction of cells. Quantitation of gene expression in a cell show similar amounts of inhibition at the level of accumulation of target mRNA or translation of target protein.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

siRNA can often be a more effective therapeutic tool than other types of gene suppression due to siRNA's potent gene inhibition and ability to target receptors with a specificity can reach down to the level of single-nucleotide polymorphisms. Such specificity generally results in fewer side effects than is seen in conventional therapies, because other genes are not be affected by application of a sufficiently sequence-specific siRNA.

There are multiple ways to deliver siRNA to the appropriate target. Standard transfection techniques may be used, in which siRNA duplexes are incubated with cells of interest and then processed using standard commercially available kits. Electroporation techniques of transfection may also be appropriate. Cells or organisms can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

For therapeutic uses, tissue-targeted nanoparticles may serve as a delivery vehicle for siRNA These nanoparticles carry the siRNA exposed on the surface, which is then available to bind to the target gene to be silenced. Schiffelers, et al., Nucleic Acids Research 2004 32(19):e149. These nanoparticles may be introduced into the cells or organisms using the above described techniques already known in the art. RGD peptides have been shown to be effective at targeting the neovasculature that accompanies the growth of tumors. Designing the appropriate nanoparticles for a particular illness is a matter of determining the appropriate targets for the particular disease. In the case of HIV, the present invention has already revealed potential targets for this powerful therapy such as the cytoplasmic domain of the CD4 receptor.

Other delivery vehicles for therapeutic uses in humans include pharmaceutical compositions, intracellular injection, and intravenous introduction into the vascular system. Inhibition of gene expression can be confirmed by using biochemical techniques such as RNA solution hybridization, nuclease protection, Northern hybridization, reverse transcription, gene expression monitoring with a microarray, antibody binding, ELISA, Western blotting, radioimmunoassay (RIA), other immunoassays, and fluorescence activated cell analysis (FACS). For RNA-mediated inhibition in a cell line or whole organism, gene expression may be assayed using a reporter or drug resistance gene whose protein product can be easily detected and quantified. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. These techniques are well known and easily practiced by those skilled in the art. For in vivo use in humans, reduction or elimination of symptoms of illness will confirm inhibition of the target gene expression.

Kits, Research Reagents, Diagnostics, and Therapeutics

In various embodiments, the present invention can also involve kits. Such kits can include pharmaceutical compositions and, in addition in certain embodiments, instructions for administration. When supplied as a kit, the different components of the composition can be packaged in separate containers and admixed immediately before use. Such packaging of the components separately can, in certain instances, permit long-term storage without losing activity of the components. In addition, if more than one route of administration is intended or more than one schedule for administration is intended, the different components can be packages separately and not mixed prior to use. In various embodiments, the different components can be packaged in one composition for administration together.

Kits may also include reagents in separate containers such as, for example, sterile water or saline to be added to lyophilized active component packaged separately. For example, sealed glass ampules may contain lyophilized biologically active materials and, in a separate ampule, sterile water, sterile saline, or other material may be provided, each of which has been packaged under a neutral non-reacting gas, such as nitrogen. Ampules may consist of any suitable material, such as glass, organic polymers such as polycarbonate, polystyrene, ceramic, metal, or any other material or combination of materials typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that, upon removal, permits the components to mix. Removable membranes may be glass, plastic, rubber, and the like.

In certain embodiments, kits can be supplied with instructional materials. Instruction may be printed on paper or other substrate, and/or may be supplied as an electronic-readable medium, such as floppy disc, mini-CD-ROM, CD-ROM, DVD-ROM, Zip disc, videotape, audio tape, and the like. Detailed instructions may not be physically associated with the kit; instead, a user may be directed to an internet web site specified by the manufacturer or distributor of the kit, or supplied as electronic mail.

The CD4 polynucleotides and CD4 proteins identified herein can be utilized for diagnostics, therapeutics, prophylaxis, as research reagents and kits. Furthermore, antisense nucleic acids, ribozymes, triplex-forming oligonucleotides, siRNAs, probes, primers, and the like may be provided in a kit.

For use in kits and diagnostics, the CD4 polynucleotides and proteins of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more CD4 polynucleotides are compared to control cells or tissues not treated with antisense HIV polynucleotides and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), fluorescent in situ hybridization (FISH) techniques (Going and Gusterson, Eur. J. Cancer, 1999, 35, 1895-904) and mass spectrometry methods (To, Comb. Chem. High Throughput Screen, 2000, 3, 235-41).

Further, the present invention provides a kit for determining the progression of HIV or AIDS. The kit comprises an CD4-specific antibody, whereby the detection of cellular CD4 levels can be carried out using the antibody in an assay as described above. The kit may comprise first and second antibodies specific to one or more epitopes of CD4, e.g., a portion of the cytoplasmic domain. The second antibody is preferably capable of binding to a conjugate of the CD4 receptor and the first antibody. For this purpose, for example, an antibody that recognizes an epitope different from that recognized by the first antibody may be used as the second antibody. It is preferable that the first and second antibodies be monoclonal antibodies. Using the kit of the present invention, the extent of CD4 down-modulation in a mammal infected with HIV can be determined, thus providing information regarding the progression of the infection.

The kit of the present invention may further comprise a substance and/or a device suitable for the detection of antibodies, the immobilization of antibodies, and the like. To immobilize the antibodies, the kit may further comprise a carrier (e.g., a microtiter plate), a solution for the immobilization (e.g., carbonate buffer) and a blocking solution (e.g., gelatin-containing phosphate buffered saline (PBS)). For the detection of the antibodies, the antibodies may be labeled previously. In this case, the kit may further comprise a detecting reagent for detecting the label. For example, when biotin is used as the labeling substance, the detecting reagent may comprise a conjugate of streptavidin with horseradish peroxidase (HRP) as well as a color-developing solution that is capable of developing a color by the action of HRP.

The present invention also provides a kit for screening compounds for activity relating to inhibition of HIV-induced CD4 down-modulation. Such a kit may utilize a cell fusion assay to identify such compounds as disclosed herein. In one embodiment, CD4-positive cells such as SupT1 cells carrying a gene encoding beta-lactamase may be provided in an ampule or other container, and HIV-1 Env-positive cells such as 293T cells may be provided in a separate ampule or other container. Reagents necessary for labeling of the HIV-1 Env-positive cells with CCF2-AM may also be provided or, alternatively, the HIV-1 Env-positive cells may be provided already labeled with CCF2-AM. Finally, buffers or other reagents necessary to carry out fusion of the two cell types provided may also be provided with the kit.

Pharmaceutical Preparations and Methods of Administration

The identified compositions may be used to treat, inhibit, control and/or prevent, or at least partially arrest or partially prevent, HIV infection or AIDS and can be administered to a subject at therapeutically effective doses for the inhibition, prevention, prophylaxis or therapy for damage caused by HIV infection or AIDS. The compositions of the present invention comprise a therapeutically effective dosage of an antibody, antisense nucleic acid, a ribozyme, a triplex-forming oligonucleotide, a siRNA, a probe, a primer, and any combination thereof, and other compounds that suppress the down-modulation of CD4 receptor protein, a term which includes therapeutically, inhibitory, preventive and prophylactically effective doses of the compositions of the present invention and is more particularly defined below. Without being bound to any particular theory, applicants surmise that these pharmaceutical compositions prevent damage caused by HIV infection and AIDS when administered to a subject suffering from a related condition by inhibiting interaction of HIV gene products with a cytoplasmic domain of the CD4 receptor, thereby inhibiting the ability of HIV to down-modulate CD4. The subject is preferably mammalian, including, but not limited to, horses, cows, dogs, cats, rats, sheep, pigs, apes, monkeys, and chickens, and most preferably human.

Therapeutically Effective Dosage

Toxicity and therapeutic efficacy of such compositions can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED₅₀ with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the IC₅₀ (the concentration of the test composition that achieves a half-maximal inhibition of symptoms) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage regimen for treating a disease or condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regimen actually employed may vary widely from subject to subject.

Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. The individual compositions may also be administered in combination with one or more additional compositions of the present invention and/or together with other biologically active or biologically inert agents (“composition combinations”). Such biologically active or inert agents may be in fluid or mechanical communication with the composition(s) or attached to the composition(s) by ionic, covalent, Van der Waals, hydrophobic, hydrophillic or other physical forces. It is preferred that administration is localized in a subject, but administration may also be systemic.

The compositions or composition combinations may be formulated by any conventional manner using one or more pharmaceutically acceptable carriers and/or excipients. Thus, the compositions and their pharmaceutically acceptable salts, esters and solvates may be specifically formulated for administration, e.g., by inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in REMINGTON'S PHARMACEUTICAL SCIENCES (A. R. Gennaro, Ed.), 20th edition, Williams & Wilkins Pa., USA (2000).

The compositions may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

Parenteral Administration

The composition or composition combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The composition may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the parenteral preparation.

Alternatively, the composition may be in powder form for constitution with a suitable vehicle, such as sterile pyrogen-free water, before use. For example, a composition suitable for parenteral administration may comprise a sterile isotonic saline solution containing between 0.1 percent and 90 percent weight per volume of the composition or composition combination. By way of example, a solution may contain from about 5 percent to about 20 percent, more preferably from about 5 percent to about 17 percent, more preferably from about 8 to about 14 percent, and still more preferably about 10 percent of the composition. The solution or powder preparation may also include a solubilizing agent and a local anesthetic such as lignocaine to ease pain at the site of the injection. Other methods of parenteral delivery of compositions will be known to the skilled artisan and are within the scope of the invention.

Oral Administration

For oral administration, the composition or composition combination may take the form of tablets or capsules prepared by conventional means with pharmaceutically acceptable excipients such as binding agents, fillers, lubricants and disintegrants:

A. Binding Agents

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation.

B. Fillers

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

C. Lubricants

Lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

D. Disintegrants

Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

The tablets or capsules may optionally be coated by methods well known in the art. If binders and/or fillers are used with the compositions of the invention, they are typically formulated as about 50 to about 99 weight percent of the composition. Preferably, about 0.5 to about 15 weight percent of disintegrant, preferably about 1 to about 5 weight percent of disintegrant, may be used in the composition. A lubricant may optionally be added, typically in an amount of less than about 1 weight percent of the composition. Techniques and pharmaceutically acceptable additives for making solid oral dosage forms are described in Marshall, Solid Oral Dosage Forms, Modern Pharmaceutics (Banker and Rhodes, Eds.), 7:359-427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may also be formulated to achieve controlled release of the composition. Oral formulations preferably contain 10% to 95% composition. In addition, the compositions of the present invention may be formulated for buccal administration in the form of tablets or lozenges formulated in a conventional manner. Other methods of oral delivery of compositions will be known to the skilled artisan and are within the scope of the invention.

Controlled-Release Administration

Controlled-release (or sustained-release) preparations may be formulated to extend the activity of the composition or composition combination and reduce dosage frequency. Controlled-release preparations can also be used to effect the time of onset of action or other characteristics, such as blood levels of the composition, and consequently affect the occurrence of side effects.

Controlled-release preparations may be designed to initially release an amount of a composition that produces the desired therapeutic effect, and gradually and continually release other amounts of the composition to maintain the level of therapeutic effect over an extended period of time. In order to maintain a near-constant level of a composition in the body, the composition could be released from the dosage form at a rate that will replace the amount of composition being metabolized and/or excreted from the body. The controlled-release of a composition may be stimulated by various inducers, e.g., change in pH, change in temperature, enzymes, water, or other physiological conditions or molecules.

Controlled-release systems may include, for example, an infusion pump which may be used to administer the composition in a manner similar to that used for delivering insulin or chemotherapy to specific organs or tumors. Typically, using such a system, the composition is administered in combination with a biodegradable, biocompatible polymeric implant that releases the composition over a controlled period of time at a selected site. Examples of polymeric materials include polyanhydrides, polyorthoesters, polyglycolic acid, polylactic acid, polyethylene vinyl acetate, and copolymers and blends thereof. In addition, a controlled release system can be placed in proximity of a therapeutic target, thus requiring only a fraction of a systemic dosage.

The compositions of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compositions will be known to the skilled artisan and are within the scope of the invention.

Inhalation Administration

The composition or composition combination may also be administered directly to the lung by inhalation. For administration by inhalation, a composition may be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas may be used to deliver a composition directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device may be used to administer a composition to the lung. DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound and a suitable powder base such as lactose or starch for these systems.

Another type of device that may be used to deliver a composition to the lung is a liquid spray device supplied, for example, by Aradigm Corporation. Liquid spray systems use extremely small nozzle holes to aerosolize liquid composition formulations that may then be directly inhaled into the lung. For example, a nebulizer device may be used to deliver a composition to the lung. Nebulizers create aerosols from liquid composition formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another example, an electrohydrodynamic (“EHD”) aerosol device may be used to deliver a composition to the lung. EHD aerosol devices use electrical energy to aerosolize liquid composition solutions or suspensions. The electrochemical properties of the composition formulation are important parameters to optimize when delivering this composition to the lung with an EHD aerosol device. Such optimization is routinely performed by one of skill in the art. Other methods of intra-pulmonary delivery of compositions will be known to the skilled artisan and are within the scope of the invention.

Liquid composition formulations suitable for use with nebulizers and liquid spray devices and EHD aerosol devices will typically include the composition with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the composition. For example, this material may be a liquid such as an alcohol, glycol, polyglycol or a fatty acid. Other methods of formulating liquid composition solutions or suspensions suitable for use in aerosol devices are known to those of skill in the art.

Depot Administration

The composition or composition combination may also be formulated as a depot preparation. Such long-acting formulations may be administered by implantation (e.g., subcutaneously or intramuscularly) or by intramuscular injection. Accordingly, the compositions may be formulated with suitable polymeric or hydrophobic materials such as an emulsion in an acceptable oil or ion exchange resins, or as sparingly soluble derivatives such as a sparingly soluble salt. Other methods of depot delivery of compositions will be known to the skilled artisan and are within the scope of the invention.

Other Systems of Administration

Various other delivery systems are known in the art and can be used to administer the compositions of the invention. Moreover, these and other delivery systems may be combined and/or modified to optimize the administration of the compositions of the present invention. Exemplary formulations using the compositions of the present invention are described below (the compositions of the present invention are indicated as the active ingredient, but those of skill in the art will recognize that pro-drugs and composition combinations are also meant to be encompassed by this term).

Without further elaboration, it is believed that one skilled in the art can, using the preceding description, utilize the present invention to its fullest extent. The specific examples below are offered by way of illustration and not by way of limiting the remaining disclosure. Prior to the examples, a description of the preparation of materials used in the examples is provided, as well as details about physical processes used in the examples and to analyze experimental results obtained in the examples.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Inhibiting CD4 Down-Modulation

A. Cells and Cell Expansion

293T, MAGIC5 and MAGIC5B cells were grown in Dulbecco's modified Eagle's medium (DMEM) supplemented with 10% fetal calf serum (FCS). MAGIC-5 and MAGIC-5B cells are CD4-positive CCR5-positive derivatives of HeLa cells. These cells contain an integrated copy of the beta-galactosidase gene under the control of the HIV-1 LTR promoter. MAGIC5B cells express CD4 receptor levels 12-fold higher than MAGIC5. Transformed T cell lines (SupT1, C8166, and Jurkat Low-CD4) were maintained in RPMI 1640 medium supplemented with 10% fetal calf serum (FCS). Ficoll-purified peripheral blood mononuclear cells (PBMC) were isolated from healthy donors and cultured in RPMI 1640 medium supplemented with 10% human AB serum. PBMC were activated with 5 μg/ml phytohaemagglutinin (PHA) (Sigma) for 2 days prior to transduction with lentiviral vectors or infection with HIV-1. After PHA stimulation cells were maintained in RPMI 1640 medium supplemented with 10% human AB serum containing 50 units IL-2/ml. All culture media used here were supplemented with 100 units penicillin/ml, 100 μg streptomycin/ml, 1 mM sodium pyruvate, and 2 mM glutamine.

B. Construction of Lentiviral Vectors Expressing Modified CD4 Molecules

To engineer lentiviral constructs expressing CD4 variants a PCR-based approach was utilized to amplify either full-length CD4 (CD4WT), or CD4 lacking its cytoplasmic domain (CD4-Δcyt). The latter construct expresses a truncated CD4 protein in which the transmembrane domain of wild-type CD4 (ending with Val at position 395) is followed by a short 6-amino acid cytoplasmic domain (sequence GSWPAS). Similarly, a CD4 chimera was engineered in which the extracellular and transmembrane domains of CD4 were fused to the HIV-1 MA protein (CD4-MA). The CD4 variants were PCR-amplified and cloned into the lentiviral transfer vectors, pHR′-CMV-GFP-WPRE, pWPI (SEQ ID NO: 5), and PPT-PGK-GFP (SEQ ID NO: 6). PPT-PGK-GFP is a lentiviral vector with a chimeric Rous sarcoma virus (RSV)-HIV 5′-LTR, and a deletion in the 3′-LTR that renders the vector self-inactivating. Expression of the transgene is driven by the human phosphoglycerate kinase (PGK) promoter. pWPI is also a SIN lentiviral vector in which the gene of interest is transcribed from an internal human EF1-alpha promoter. The transcribed unit also contains an internal ribosome entry site (IRES) element from EMCV that allows internal initiation of translation of GFP. In pHR′-CMV-CD4-WPRE, the transgene is expressed from the internal human CMV immediate early promoter. Unlike PPT-PGK-CD4 and pWPI, pHR′-CMV-CD4-WPRE can be mobilized upon infection with replication-competent HIV virus. All lentiviral vectors used here contain a post-transcriptional regulatory element from the woodchuck hepatitis virus (WPRE) that enhances expression of transgenes.

C. Vector Particle Production

For preparation of HIV-based lentiviral vector particles coated with the vesicular stomatitis virus glycoprotein (VSV G) a previously described three-plasmid cotransfection procedure was used. 293 T cells were co-transfected with 10 μg of CD4 lentiviral vector (PPT-, pWPI-, or pHR′-based), together with 10 μg of a packaging construct (pCMVΔR8.91), and 2.5 μg of a plasmid encoding VSV G (pMD.G). Forty-eight hours later vector particles were collected from culture supernatants, filtered through 0.45 um nitrocellulose membranes, aliquoted, and frozen to −80° C. until use. All transductions described here were performed with unpurified vector-containing supernatants from 293T cells.

D. Transduction with Lentiviral Vectors and Viral Replication Assays

293T, MAGIC-5B, SUPT-1, C8166, and PBMC cells were transduced following a spinoculation procedure. Briefly, 0.5×106 cells were incubated with viral or vector supernatants (200 to 500 ng p24 antigen) in 24-well plates in the presence of 4 μg/ml polybrene and 10 mM Hepes (pH 7.4). Mixtures of cells and vectors were centrifuged at room temperature for 90 min (2,500 rpm) in a table-top centrifuge (Sorvall RT6000B). After centrifugation, cells were washed with propagation medium and then incubated in the same medium for two to three days at 37° C. Transduced cells were infected with HIV-1 (1 μg p24 protein) following the above protocol. Viral replication assays in PBMC were performed by incubating cells with supernatant containing viruses in the absence of polybrene. Routinely, PBMC were activated for 48 hours with 5 μg/ml PHA, transduced with lentiviral vectors and sorted for GFP expression 2-3 days later. 3-4 days after sorting, 1×10⁶ transduced PBMC were infected with 2,000 TCID50 (without additional PHA treatment) in 150 μl of propagation media. After 2 hours at 37° C., cells were washed twice and distributed into 96-well plates (2×105/well). Cells were maintained in the presence of 50 units IL-2/ml. Replication assays in Jurkat Low-CD4 cells were performed by incubating 1×10⁶ cells with HIV-1 R9 (50 ng p24).

E. HIV-1 Virus Production

Infectious HIV-1 (R9) was produced by transient transfection of 293T cells with HIV-1 proviral clones. VSV G-pseudotyped R9HIV-1 was generated by co-transfecting proviral DNA (10-20 μg) with 2.5 μg of pMD.G plasmid DNA. The CCR5-tropic HIV-1 BaL virus was harvested and purified from the supernatant of infected monocyte-derived macrophages.

F. Single-Round Infectivity Assays

Infectivity assays were performed with MAGIC-5 cells using equal amounts of input virus (measured as ng p24 protein) in the presence of 20 μg/ml DEAE dextran. Infection was allowed for 48 hours. Routinely, after 8 hrs of infection, 1 μM AZT was added to block second rounds of virus replication. Infected cells were scored after staining for beta-galactosidase with X-Gal.

G. Protein Analysis

Incorporation of cellular and viral proteins in HIV particles was determined by western-blot with the following antibodies (NIH AIDS Research Reference Reagent Program): for HIV-1 Env, a gp120-specific sheep antiserum was used; CD4 was probed with the T4-4 rabbit antiserum; gp41 with the 2F5 human mAb; and p24 was analyzed with the 183-H12-5C murine mAb. Loading controls for cell lysates were performed by probing with an α-tubulin mAb (Sigma). Virion-associated proteins were pelleted through a 20% sucrose cushion at 26,000 rpm in a SW28 rotor (Beckman) for 1.5 h at 4° C. Equal amounts of p24 protein were separated onto 10% SDS-polyacrylamide gel, transferred to polyvinylidene difluoride membrane and then probed with specific antibodies. Antibody binding was detected by enhanced chemiluminescence (ECL, Western Blotting Kit, Amersham) using horseradish peroxidase-conjugated secondary antibodies (Amersham Bioscience, Inc.).

H. Flow Cytometry and Cell Sorting

Surface staining of CD4 was performed with either OKT4 mAb, followed by addition of goat anti-mouse Cy-5 conjugated antibody, or with CD4-V4 mAb (PE-conjugated, Becton Dickinson). The epitopes recognized by these mAbs do not overlap with the gp120-binding domain. Intracellular staining of HIV-1 Gag was performed with a p24-specific mAb (FITC-conjugated KC57 clone, Coulter) using a cell permeabilization kit following the recommendations of the manufacturer (Caltag Lab). PBMC transduced with lentiviral vectors were sorted for enrichment of GFP-positive/CD4-positive cells. PBMC cells were resuspended at 20×10⁶ cells/ml in PBS with 2% FCS, and stained with a CD4 mAb (CD4-PE-Cy5 conjugated, DAKO) at room temperature for 15 min. Then, cells were washed twice and resuspended in RPMI supplemented with 5% human AB serum 10-25×10⁶ cells/ml. Cells were immediately sorted with a FACSVantage SE system (Becton Dickinson), collected in RPMI supplemented with 20% human AB serum, centrifuged and resuspended in propagation media supplemented with 50 units IL-2/ml.

### Example 2

### Lentiviral Vectors Express CD4 Molecules Resistant to Down-Modulation by HIV-1 Nef and Vpu

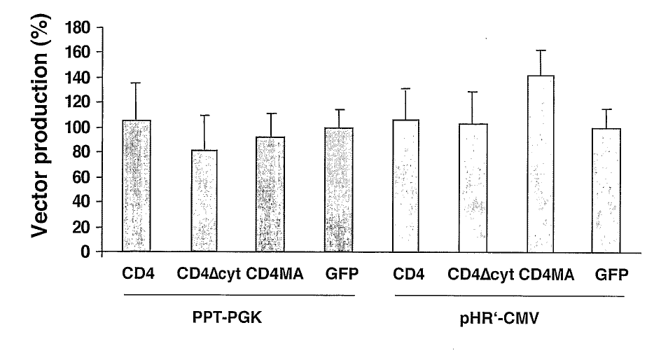

Lentiviral vectors can be used to deliver to a cell full length (wild-type) CD4, CD4 lacking its cytoplasmic domain (CD4Δcyt), or the extracellular and transmembrane domains of CD4 fused to the full-length HIV-1 Matrix protein (CD4-MA). The engineered proteins can be produced in 293T cells transduced with the appropriate lentiviral vector. The ability of the various CD4 proteins to be down-modulated by HIV-1 Nef can be tested by transfecting these same 293T cells with plasmids expressing either the HIV-1 NA7 primary nef allele fused to GFP (Nef-GFP), or GFP alone. In one such experiment, expression of Nef-GFP decreased 15-fold the surface levels of full length CD4, but did not alter the expression of CD4Δcyt or CD4-MA. This result was consistent with the requirement of the CD4 cytoplasmic domain for efficient Nef-induced down-modulation. Expression of CD4 as described above does not significantly affect the level of vector production. This conclusion was arrived at by testing the amount of viral p24 protein in cultures of 293T producer cells. These cells were co-transfected with packaging and VSV G plasmids, together with either control transfer vectors (GFP), or vectors expressing the CD4 variants described above. No significant reduction in p24 was observed with any of the vectors (PPT-PGK- or pHR′-CMV-based), indicating that expression of the various CD4 transgenes does not interfere with vector production in 293T cells. The results are provided in FIG. 4.

### Example 3

### Suppression of HIV-1 Infectivity by Lentiviral Vectors Expressing Truncated CD4 Proteins

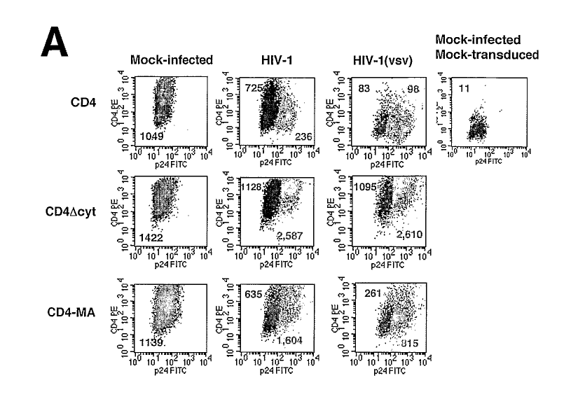

Interference with HIV-1 infectivity can be analyzed in CD4-negative 293T cells. When transduced with CD4 lentiviral vectors, these cells become permissive to HIV-1. This strategy ensures that release of HIV-1 particles occurs only from the lentiviral vector-transduced cells. In one such analysis, 293T cells were infected with HIV R9 (1000 ng p24) following a spinoculation procedure. Other 293T cells were infected with VSV G-pseudotyped HIV-1 particles (HIV-1 (VSV)), which infects cells to a higher extent than HIV-1 particles using the natural viral envelope (Env) for entry. As shown in FIG. 5, as estimated by surface staining with antibodies, more than 90% of cells were successfully transduced and expressed CD4. Upon infection with HIV-1, full-length CD4 was efficiently down-modulated in p24-positive cells (see top middle panel of FIG. 5). In contrast, however, the levels of receptor remained elevated in HIV-1 infected cells transduced with CD4Δcyt and CD4-MA, having surface levels 11- and 7-fold higher, respectively, than those observed in infected cells transduced with wild-type CD4. Pseudotyping HIV-1 with VSV G resulted in enhanced infection (4 to 5 fold higher, estimated as a percentage of p24-positive cells), as well as 52% higher levels or p24 expression in infected cells. Consequently, the level of down-modulation of full-length CD4 occurred to higher extents. Surface levels of CD4-MA were partially reduced as compared to cells infected with HIV-1. CD4Δcyt molecules, however, remained completely resistant to down-modulation by HIV-1 (VSV) (see right panel of FIG. 5). Thus, the lentiviral vector system used achieved elevated levels of CD4 expression. Further, expression of Env in HIV-1 infected cells is not sufficient to down-modulate receptor molecules insensitive to the action of Nef and Vpu.

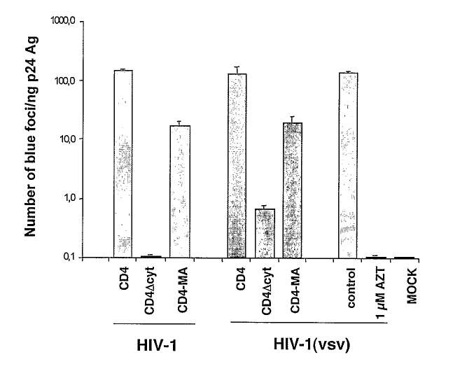

293 T cells transduced with CD4 vectors and infected with HIV-1 did not show significant differences in the release of viral particles, as estimated by p24 ELISA, as compared to mock-transduced cells. The infectivity of HIV-1 particles produced from 293T transduced cells can be analyzed in single-round infectivity assays in MAGIC5 cells. Such an analysis was undertaken, with MAGIC5 cells challenged with equal amounts of p24 and the number of blue foci estimated. The results are provided in FIG. 6. Lentiviral-based expression of CD4Δcyt reduced the infectivity of HIV-1 more than 1000-fold. The extent of inhibition was comparable to that observed by addition to target cells of 1 μM AZT, an inhibitor of the HIV reverse transcriptase (see FIG. 6). Thus, these lentiviral vectors are able to efficiently block HIV-1 infectivity in the presence of a full set of CD4 down-modulator genes (nef, vpu, and env). Particles synthesized in cells transduced with CD4-MA expressed lower amounts of the fusion protein on the surface of infected cells as compared to CD4Δcyt, with mean values of 1604 and 2857, respectively. The viral progeny from cells transduced with CD4Δcyt and then infected with VSV G-pseudotyped HIV-1 unexpectedly showed severely reduced infectivity (100-fold), indicating that higher levels of viral products were not sufficient to overcome the inhibitory effects of truncated CD4. Expression of full-length CD4 by itself did not affect HIV infectivity, indicating that the inhibitory effects mediated by CD4Δcyt and CD4-MA are likely due to expression of transgenes and not to the lentiviral vectors, which might interfere with HIV replication at different stages.

### Example 4

### Cytoplasmic Domain-Deficient CD4 Negatively Interferes with HIV-Induced Down-Modulation of Full-Length CD4

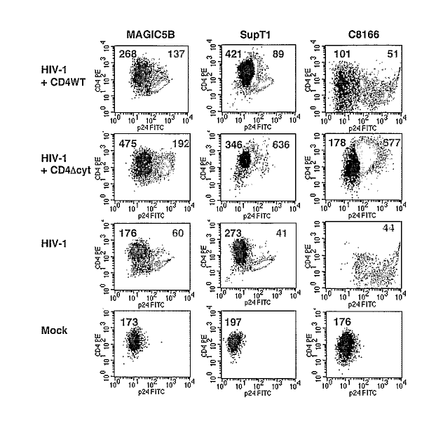

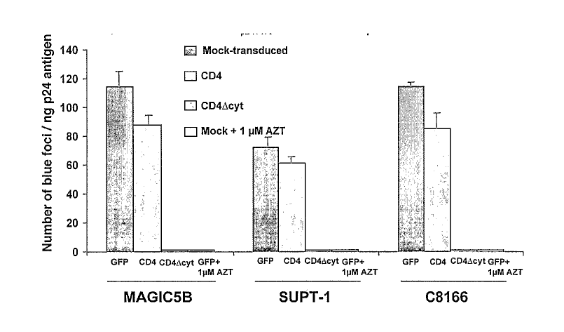

Truncated receptor provided to a cell via one of the above-described vectors negatively interferes with HIV-induced down-regulation of endogenous CD4 receptor. To demonstrate this, several CD4-positive cell lines permissive to HIV-1 infection were used. Though any suitable cell line could be used, the three chosen were: SupT1, C8166, and MAGIC5B. These were selected because they express high levels of viral receptor, as shown in the bottom panel of FIG. 7. Transduction efficiencies with GFP control vectors were higher than 90% in all cell lines, as estimated by flow cytometry. Infection of mock-transduced cells with HIV-1 led to efficient down-modulation of CD4. Receptor down-modulation was also efficiently achieved in cultures transduced with full-length CD4. In these cultures, a population of low-CD4/p24-positive infected cells that had undergone receptor down-modulation emerged. By contrast, infection of cells previously transduced with CD4Δcyt failed to down-modulate the receptor, which maintained surface levels approximately 3- to 4-fold higher than those observed in mock-infected cells. Unexpectedly, p24-positive cells with low levels of CD4 receptor were no longer observed in cultures of SupT1 and C8166 cells transduced with CD4Δcyt, indicating that expression of truncated receptor interferes with down-modulation of endogenous full-length CD4 in lymphoblastoid cells. The infectivity of viral particles released from the cells was determined as with respect to FIG. 5, above. Overexpression of the full-length CD4 led to a small reduction in infectivity in three cell types, as compared to cells transduced with control GFP vectors. In contrast, cells transduced with truncated CD4 produced viral particles with largely diminished infectivity, as indicated in FIG. 8. Mobilization of pHR′-CMV vectors by HIV-1 did not significantly contribute to the inhibiting effect observed in the above experimental settings, since similar results were observed in SupT1 cells transduced with the self-inactivating PPT-PGK lentiviral vector expressing CD4Δcyt (see FIG. 8.).

### Example 5

### Inhibition of HIV-1 Replication in Primary Lymphocytes

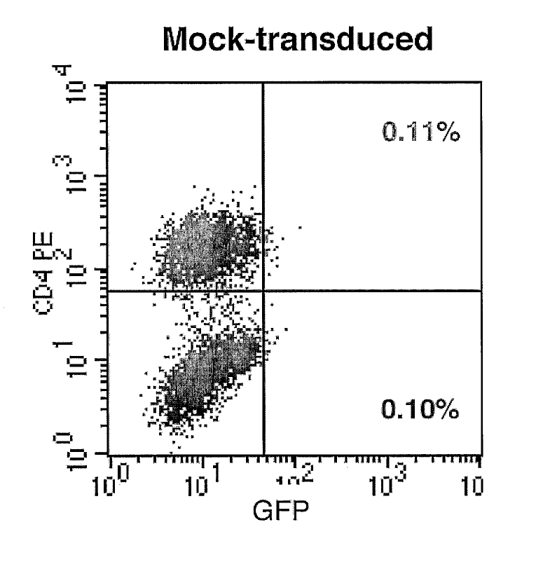

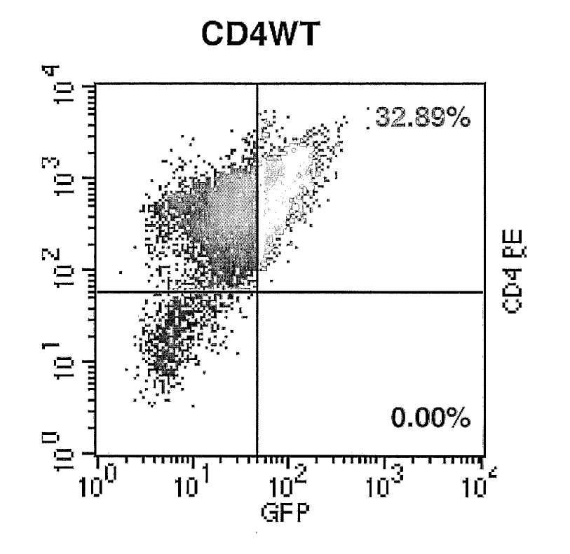

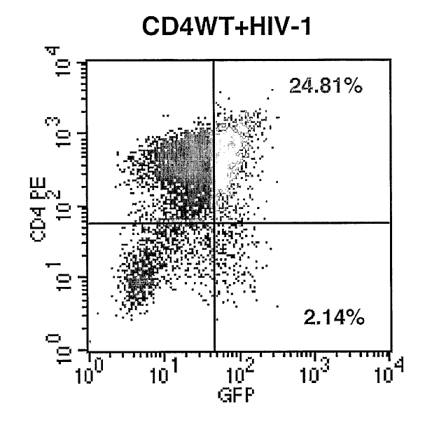

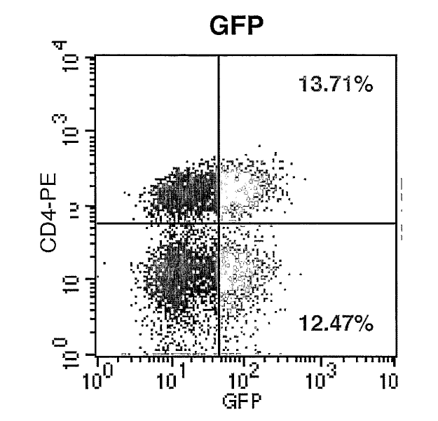

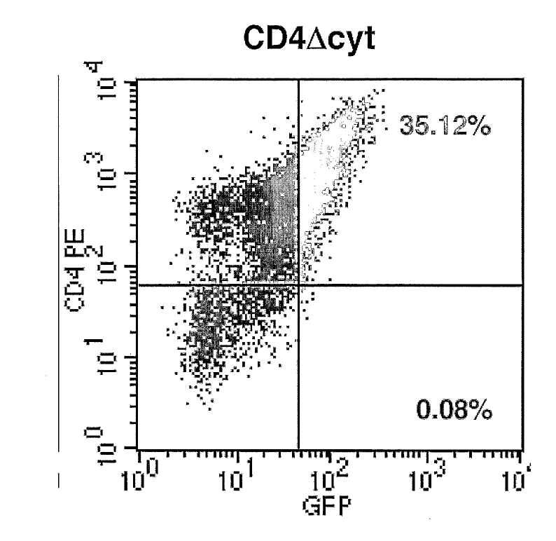

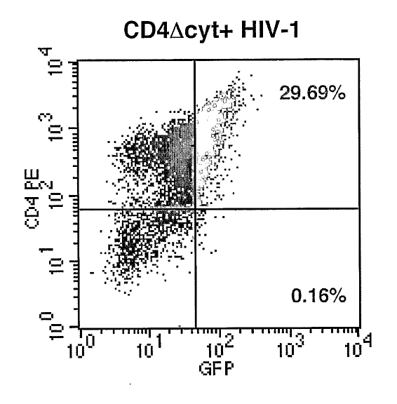

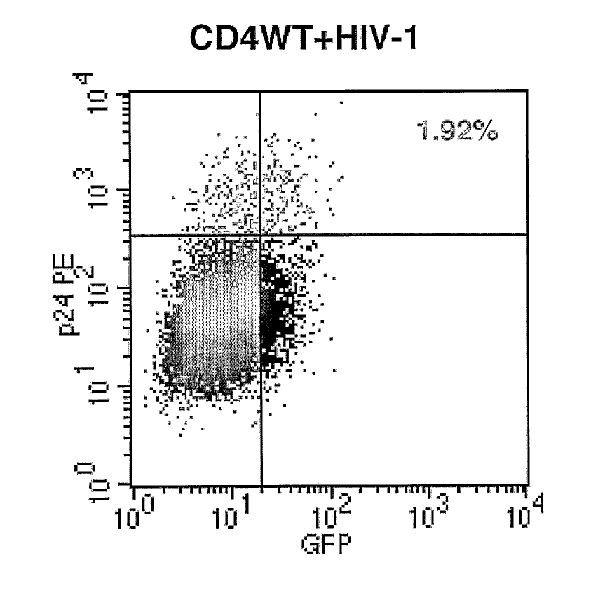

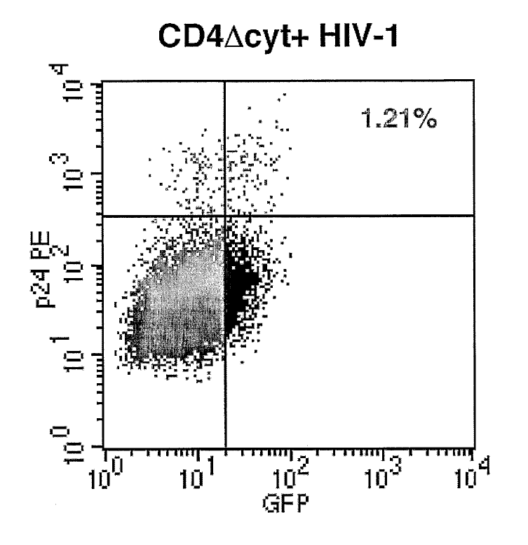

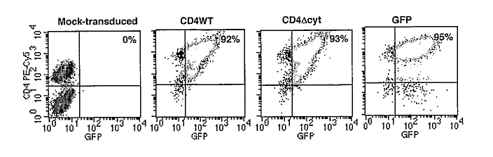

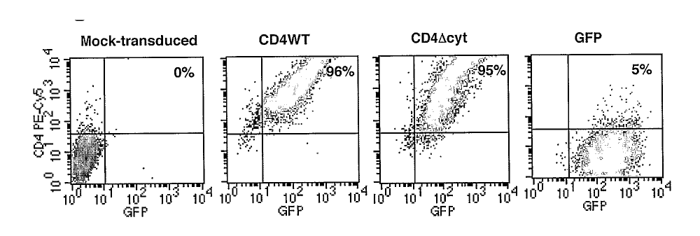

As compared to transformed T cells, as described above, treatment of PBMC with lentiviral vectors results in transduction efficiencies of only 20%-40%, as estimated by the number of GFP-positive cells by flow cytometry (see FIGS. 9-16). Thus, CD4 lentiviral vectors derived from pWPI were used for transduction of PBMC. In addition to the transgene, cells transduced with these vectors also express GFP under the control of an IRES element. Transduced cells can thus be readily identified by gating GFP-positive cells. Primary cells were transduced either with GFP alone, full-length CD4, or with the most potent inhibitor of HIV infectivity, CD4Δcyt. Three days after transduction with bicistronic vectors, PBMCs were infected with HIV-1 (R9) and analyzed by flow cytometry for surface CD4 and GFP fluorescence (see FIG. 17). Infection with HIV-1 resulted in the emergence of a population of GFP-positive cells that had undergone CD4 down-modulation (see FIG. 19, 2.14% of the culture). In contrast, PBMC transduced to similar levels with a CD4Δcyt lentiviral vector resulted in reduced numbers of low-CD4/GFP-positive cells. These results indicate that CD4Δcyt is resistant to down-modulation by primary T cells infected with wild-type HIV-1, and also that the truncated receptor interferes with the removal of endogenous full-length CD4 from the cell surface. The possibility that truncated CD4 interferes with the expression of HIV proteins can be ruled out by staining aliquots of the cell cultures intracellularly with antibodies specific for HIV-1 p24. Doing so revealed that 1.92% of the cells transduced with full-length CD4 and infected with HIV stain positive for p24 antigen. This value is comparable to the fraction of cells that underwent CD4 down-modulation in the same culture (2.14%). This difference cannot, however, account for the more than 10-fold reduction observed in CD4 down-modulation, as compared to cells transduced with full-length receptor. This reduction in the number of p24-positive cells is likely due to a block in second rounds of infection, since the output virus produced under such conditions in which CD4 down-modulation is blocked is unable to infect new target cells. The experimental results above, therefore, indicate that the expression of CD4 in primary lymphocytes interferes with the down-modulation of endogenous full-length receptor in HIV-1 infected cells. This interference occurs without affecting the steps of the viral life cycle that lead to expression of the late Gag viral protein.

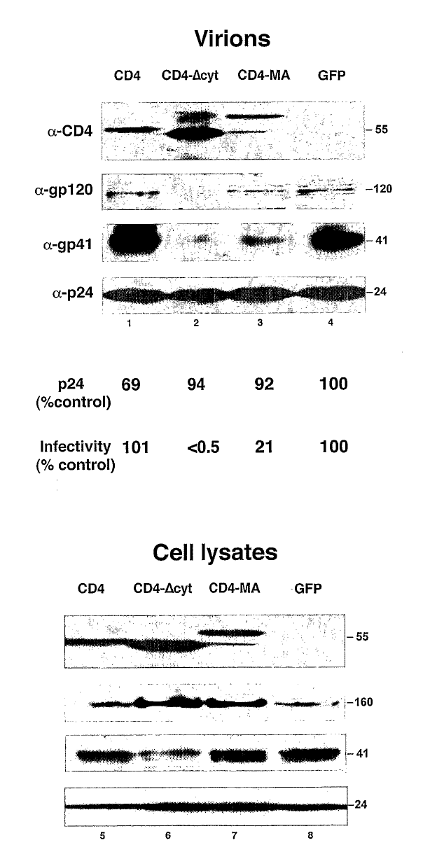

Viral replication assays were performed with transduced primary lymphocytes. Cultures of PBMCs were incubated with bicistronic vectors and enriched in transduced cells by sorting for GFP expression. This procedure resulted in cultures containing ˜90% GFP-positive cells (see FIG. 21). Enriched cultures were challenged with the CCR5-tropic HIV-1 BaL strain. The extent of viral replication, estimated as the amount of p24 antigen in cultures, is shown in FIG. 21. Transduction with full-length CD4 resulted in faster replication profiles than those observed in cells transduced with control vectors (GFP alone). This may be due to the enhancement in HIV entry promoted by the elevated levels of receptor. Conversely, cells transduced with CD4 molecules resistant to the action of Nef and Vpu showed severely reduced HIV-1 replication. The residual level of HIV-1 replication observed in these cultures may be due to viral production from the fraction of untransduced cells still present in the enriched cultures (5-10%). Viral replication assays were also performed in Jurkat Low-CD4 cells infected with the CXCR4-tropic R9 strain (FIG. 21). Enrichment of these cultures was not necessary, since treatment with lentiviral vectors resulted in transduction efficiencies higher than 95%, estimated as percentage of GFP-positive cells (FIG. 21). As shown in primary cells, transduction of Jurkat cells with full-length CD4 resulted in faster viral replication. This enhancement was more pronounced than the one observed in primary cells, probably due to the much lower levels of CD4 receptor present in Jurkat Low-CD4 cells (data not shown). Expression of truncated CD4 resulted in complete abrogation of HIV-1 replication. Of note, revertant viruses were not detected after more than 60 days in two independent experiments in which Jurkat Low-CD4 cells were transduced with CD4Δcyt vectors and exposed to HIV-1. The above findings demonstrate the ability of lentiviral vectors to interfere with CD4 down-modulation and HIV-1 replication in primary cells, and highlight the potency of the observed inhibitory effect.

### Example 6

### Mechanism of Inhibition of HIV-1 Infectivity

To address the mechanism of inhibition of HIV-1 replication mediated by CD4 lentiviral vectors, transduced 293T cells were infected with HIV-1 pseudotyped with VSV G. The extent of inhibition mediated by CD4Δcyt estimated in single-round infectivity assays was greater than 99%. Viral particles were purified through sucrose cushions and analyzed by western-blot with antibodies specific for CD4, gp120, gp41, and p24. In parallel, total cell lysates of the producer cells were analyzed (FIG. 6). Expression of CD4 molecules lacking the cytoplasmic domain (CD4Δcyt and CD4-MA) resulted in accumulation of a 160 kDa protein band immuno-recognized with anti-gp120 antibodies. This band was also recognized by gp41-specific antibodies (not shown) and represents the gp160 envelope precursor. Concomitantly, a reduction in the steady-state levels of gp41 was evident in cells transduced with CD4Δcyt. Virions produced in cells expressing CD4 molecules incorporated the receptor in their membranes, whereas the levels of gp120 incorporation were reduced. CD4 incorporation was proportional to gp120 exclusion, which was more severe in virions produced in the presence of CD4Δcyt, the most potent inhibitor of HIV-1 infectivity. Reductions in gp41 incorporation were also observed in membranes from virions produced in the presence of CD4Δcyt and CD4-MA. These results are in agreement with previous reports and suggest that the observed inhibitory effects are likely due to interference with incorporation of Env into viral membranes. Furthermore, CD4 molecules incorporated into viral membranes could also interfere with Env function by saturating its CD4 binding sites.

### Example 7

### CD4 Assay

CD4-positive T-cells (such as SupT1 cells) and HIV-1 Env-positive cells (such as 293T cells) may be utilized. SupT1 cells can be infected with a virus lacking env (VSVg-pseudotyped), but carrying nef and vpu, as well as a gene encoding bacterial beta-lactamase. Infected cells may be distributed in 96-well plates in which candidate inhibitors of CD4 down-modulation are to be evaluated (such inhibitors may, for example, be added 8 hours after infection). Approximately 24 to 36 hours after infection, CCF2-AM-labeled Env-positive cells may be added to the well, and fusion may then be allowed to progress for about 1 to 4 hours. Upon fusion, beta-lactamase will cleave the CCF2-AM molecule, which can be identified by emission of blue light (460 nm) upon excitation at 409 nm. Uncleaved CCF2-AM emits at 538 nm. Infected cells down-modulate the CD4 receptor and fuse at lower rates. In the presence of inhibitors of CD4 down-modulation, CD4 receptor is maintained at high levels and fusion occurs to a higher extent. Uninfected cells, or cells in which expression of the viral genome is blocked by inhibitors present in the mixture, fail to transfer beta-lactamase into fused cells and will result in negative readouts. The extent of fusion, as measured by calorimetric assay, is proportion to the degree of inhibition of HIV-induced CD4 down-modulation.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically intended to be within the scope of the present invention, and incorporated herein by reference in its entirety, is the following publication: Pham H M, Arganaraz E R, Groschel B, Trono D, Lama J, Lentiviral vectors interfering with virus-induced CD4 down-modulation potently block human immunodeficiency virus type 1 replication in primary lymphocytes, J Virol. 2004 December; 78(23):13072-81.

1-46. (canceled)

47. A method for treating HIV infection in a mammalian subject, the method comprising administering to the subject a therapeutically effective amount of a CD4 receptor down-modulation inhibitor to a subject in need thereof, wherein the inhibitor is CD4Δcyt.

48. A method according to claim 47, wherein CD4Δcyt comprises a polypeptide sequence corresponding to SEQ ID NO:1.

49. A method according to claim 47, wherein the CD4Δcyt is a CD4Δcyt fragment or CD4Δcyt variant.

50. A method according to claim 47, wherein the inhibitor is a CD4 receptor down-modulation inhibitor is selected from the group consisting of CD4 cytoplasmic domain, CD4 cytoplasmic domain fragment, and CD4 cytoplasmic domain variant.

51. A method according to claim 47, wherein the subject is a human.

52. A method for treating HIV infection in a mammalian subject, the method comprising administering to the subject a therapeutically effective amount of a vector comprising a polynucleotide encoding a CD4 receptor down-modulation inhibitor, wherein the inhibitor is CD4Δcyt.

53. A method according to claim 52, wherein the polynucleotide encoding CD4Δcyt comprises a sequence corresponding to SEQ ID NO:2.

54. A method according to claim 52, wherein the vector comprises a polynucleotide encoding a CD4Δcyt fragment or CD4Δcyt variant.

55. A method according to claim 52, wherein the vector comprises a polynucleotide encoding an inhibitor selected from the group consisting of CD4 cytoplasmic domain, CD4 cytoplasmic domain fragment, and CD4 cytoplasmic domain variant.

56. A method according to claim 52, wherein the vector is selected from the group consisting of an adenoviral vector and a lentiviral vector.

57. A method according to claim 52, wherein the subject is a human.

58. A method for identifying an activity of an inhibitor of HIV-induced down-modulation of a CD4 receptor comprising:
providing a plurality of CD4-positive cells, said CD4-positive cells being infected with HIV;
providing a target compound to be assayed for activity in inhibiting HIV-induced CD4 down-modulation;
incubating said CD4-positive cells in the presence of said target compound;
providing a plurality of HIV-1 Env-positive cells;
incubating said HIV-1 Env-positive cells with said CD4-positive cells and said target compound; and
measuring the level of fusion between said HIV-1 Env-positive cells and said CD4-positive cells, whereby a relatively higher measured level of fusion is indicative of a relatively higher activity of the target compound as an inhibitor of HIV-induced down-modulation.

59. A method according to claim 58, wherein said CD4-positive cells express a beta-lactamase enzyme, and further wherein said HIV Env-1 positive cells are labeled with a substrate of beta-lactamase.

60. A method according to claim 59, wherein said substrate of beta-lactamase is CCF2-AM.

61. A vector operably linked to a polynucleotide encoding a CD4 protein lacking a cytoplasmic domain.

62. A vector according to claim 61, wherein the vector is selected from the group consisting of an adenoviral vector and a lentiviral vector.

63. A vector according to claim 61, wherein the encoded CD4 protein lacking a cytoplasmic domain comprises a nucleotide sequence corresponding to SEQ ID NO:2.

64. A kit for screening compounds for inhibition of HIV-induced down-modulation of CD4 comprising: a plurality of CD4-positive cells, said CD4-positive cells infected with HIV; a plurality of HIV-1 Env-positive cells; and a buffer in which a compound to be screened for inhibition of HIV-1 induced CD4 down-modulation can be incubated in the presence of said CD4-positive cells, and further in which said HIV-1 Env-positive cells can be incubated in the presence of said compound to be screened and said CD4-positive cells.

65. A kit according to claim 64, further comprising a detection system for detecting the extent of cleavage of said substrate of beta-lactamase.

66. A kit according to claim 65, further comprising a detection system for determining the extent of cleavage of CCF2-AM.


SEQUENCE LISTING (6 sequences)

SEQ ID NO: 1 - protein, 426 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTTICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVGSWPAS

SEQ ID NO: 2 - DNA, 1281 bp, Homo sapiens
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcggatcctggccagctagctag

SEQ ID NO: 3 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 4 - DNA, 1377 bp, Homo sapiens
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttga

SEQ ID NO: 5 - DNA, 11101 bp, Homo sapiens
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttccgatcacgagactagcctcgaggtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgctcaagcttcgaattaattctgcagagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 6 - DNA, 7384 bp, Homo sapiens
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaagaagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctgaatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc